(19) United States
(12) Patent Application Publication
(10) Pub. No.: US 2014/0193519 A1
(43) Pub. Date: Jul. 10, 2014

(54) INDIBULIN THERAPY

(71) Applicant: ZIOPHARM ONCOLOGY, INC., Boston, MA (US)

(72) Inventors: Barbara P. Wallner, Cohasset, MA (US); Brian Eric Schwartz, Woodbridge, CT (US); Philip B. Komarnitsky, Chestnut Hill, MA (US); Gerald Bacher, Germering (DE); Bernhard Kutscher, Maintal (DE); Gerhard Raab, Ronneburg (DE)

(73) Assignee: ZIOPHARM ONCOLOGY, INC., Boston, MA (US)

(21) Appl. No.: 13/958,160

(22) Filed: Aug. 2, 2013

Related U.S. Application Data
(63) Continuation of application No. 13/681,828, filed on Nov. 20, 2012, now abandoned.
Application No. 13/681,828 is a continuation of application No. 11/986,844, filed on Nov. 27, 2007, now abandoned.
(60) Provisional application No. 60/861,454, filed on Nov. 28, 2006.
(60) Provisional application No. 60/872,874, filed on Dec. 5, 2006.
(60) Provisional application No. 60/922,268, filed on Apr. 6, 2007.
(60) Provisional application No. 60/993,829, filed on Sep. 14, 2007.
(60) Provisional application No. 61/000,158, filed on Oct. 23, 2007.

(51) Int. Cl. A61K 31/4439; A61K 31/475; A61K 31/337; A61K 31/138; A61K 31/282; A61K 33/24; A61K 31/517; A61K 31/7068; A61K 31/53; A61K 31/566; A61K 38/08; A61K 31/569; A61K 31/513; A61K 31/7048

(52) U.S. Cl. CPC A61K 31/4439; A61K 31/513; A61K 31/475; A61K 31/337; A61K 31/138; A61K 31/282; A61K 31/7048; A61K 31/517; A61K 31/7068; A61K 31/53; A61K 31/566; A61K 38/08; A61K 31/569; A61K 33/24
USPC 424/649; 514/339; 514/274; 514/283; 514/27; 514/266.4; 514/49; 514/245; 514/171; 514/19.3

(57) ABSTRACT

The invention provides combination therapy, wherein one or more other therapeutic agents are administered with indibulin or a pharmaceutically acceptable salt thereof and the combination is synergistic. Another aspect of the invention relates to the treatment of cancer with indibulin as a single agent. Another aspect of the invention relates to dosing regimen for administration of oral dosage forms of indibulin.

### CROSS REFERENCE TO RELATED APPLICATIONS

This application claims the benefit of priority to U.S. Provisional Patent Application No. 60/861,454, filed Nov. 28, 2006, U.S. Provisional Patent Application No. 60/872,874, filed Dec. 5, 2006, U.S. Provisional Patent Application No. 60/922,268, filed Apr. 6, 2007, and U.S. Provisional Patent Application No. 61/000,158, filed Oct. 23, 2007, which applications are hereby incorporated by reference in their entirety.

### BACKGROUND OF THE INVENTION

During mitosis, a cell's DNA is replicated and then divided into two new cells. The process of separating the newly replicated chromosomes into the two forming cells involves spindle fibers constructed with microtubules, which themselves are formed by long chains of smaller protein subunits called tubulins. Spindle microtubules attach to replicated chromosomes and pull one copy to each side of the dividing cell. Without these microtubules, cell division is not possible.

Microtubules therefore are among the most important sub-cellular targets of anticancer chemotherapeutics because they are present in all cells and are necessary for mitotic, interphase and cell maintenance functions (e.g. intracellular transport, development and maintenance of cell shape, cell motility, and possibly distribution of molecules on cell membranes). Compounds that interact with tubulin can interfere with the cell cycle by causing tubulin precipitation and sequestration, thereby interrupting many important biologic functions that depend on the microtubular class of subcellular organelles. Therefore, such compounds can potentially inhibit the proliferation of tumor cell lines derived from various organs. See, e.g., Bacher et al. (2001) Pure Appl. Chem. 73:9 1459-1464 and Rowinsky & Donehower (1991) Pharmac. Ther. 52:35-84.

Accordingly, new, synthetic, small-molecule chemical entities that bind to tubulin, but are neither a substrate of transmembrane pumps nor interfere with the function of axonal microtubules, would strongly increase the therapeutic index in the treatment of malignancies.

A series of synthetic molecules that bind to tubulin are currently being evaluated in the preclinical or early clinical stage. Among them is the synthetic compound, Indibulin having the following structure:

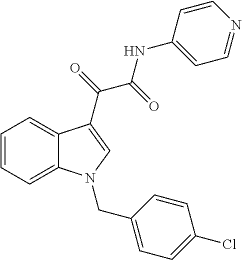 [SMILES: O=C(Nc1ccncc1)C(=O)c1cn(Cc2ccc(Cl)cc2)c2ccccc12]

Indibulin is a synthetic small molecule tubulin inhibitor with significant antitumor activity in vitro and in vivo. It destabilizes microtubules in tumor cells, as well as in a cell-free system. The binding site of indibulin does not appear to overlap with the tubulin-binding sites of the well-characterized microtubule-destabilizing agents vincristine or colchicine. Furthermore, the molecule selectively blocks cell cycle progression at metaphase. Improved methods of using indibulin to treat hyperproliferative disorders would be useful.

### SUMMARY OF THE INVENTION

In certain embodiments, the invention relates to a method for the treatment of cancer, comprising administering a indolyl-3-glyoxylic acid derivative. In certain embodiments the indolyl-3-glyoxylic acid derivative is a N-substituted indole-3-glyoxylamide or a pharmaceutically acceptable salt thereof. In certain embodiments, the indolyl-3-glyoxylic acid derivative is indibulin. In certain embodiments, the cancer is selected from adenoid cystic carcinoma, renal cell carcinoma, breast cancer, ovarian cancer, prostate cancer, vulvar cancer, glioblastoma, and lung cancer. In certain embodiments, the cancer is selected from renal cell carcinoma, breast cancer, ovarian cancer, prostate cancer, vulvar cancer, glioblastoma, and lung cancer.

In certain embodiments, the invention relates to a method for the treatment of a cancer selected from adenoid cystic carcinoma, renal cell carcinoma, breast cancer, vulvar cancer, glioblastoma, and lung cancer comprising administering an indolyl-3-glyoxylic acid derivative, preferably indibulin. In certain embodiments, the cancer is selected from renal cell carcinoma, breast cancer, vulvar cancer, glioblastoma, and lung cancer comprising administering an indolyl-3-glyoxylic acid derivative, preferably indibulin.

One aspect of the invention relates to combination therapy, wherein an indolyl-3-glyoxylic acid derivative or a pharmaceutically acceptable salt thereof is administered with one or more other therapeutic agents and the combination shows efficacy that is greater than the efficacy of either agent being administered alone (e.g., synergistic or additive antitumor effect). Such combination treatment may be achieved by way of the simultaneous, sequential, or separate dosing of the individual components of the treatment.

Another aspect of the invention provides combination therapy, wherein indibulin or a pharmaceutically acceptable salt thereof is administered with one or more other therapeutic agents and the combination shows efficacy that is greater than the efficacy of either agent being administered alone (e.g., synergistic or additive antitumor effect). Such combination treatment may be achieved by way of the simultaneous, sequential, or separate dosing of the individual components of the treatment.

In certain embodiments, the indolyl-3-glyoxylic acid derivative is conjointly administered with one or more chemotherapeutic agents, hormonal therapeutic agents, targeted therapy, radiotherapy, immunotherapy, gene therapy, or surgery, preferably a chemotherapeutic. Such conjoint therapy may be as a single formulation of the two or more agents (e.g., tablet, pill, or liquid formulation) or the agents may be formulated separately.

Another aspect of the invention relates to methods for the treatment of cancer, comprising administering an indolyl-3-glyoxylic acid derivative or a pharmaceutically acceptable salt thereof in combination with another therapeutic agent, wherein the combination shows efficacy that is greater than the efficacy of either agent being administered alone (e.g., synergistic or additive antitumor effect). In certain embodiments, the cancer is selected from breast, lung, ovarian, and prostate cancer.

Another aspect of the invention relates to methods for the treatment of cancer, comprising administering indibulin or a pharmaceutically acceptable salt thereof in combination with another therapeutic agent, wherein the combination shows efficacy that is greater than the efficacy of either agent being administered alone (e.g., synergistic or additive antitumor effect). In certain embodiments, the cancer is selected from breast, lung, ovarian, and prostate cancer.

Another aspect of the invention relates to a kit comprising an indolyl-3-glyoxylic acid derivative or a pharmaceutically acceptable salt thereof and another therapeutic agent.

Another aspect of the invention relates to a kit comprising indibulin and another therapeutic agent.

### BRIEF DESCRIPTION OF THE FIGURES

FIG. 1A shows the dose response curve of the indibulin-erlotinib combination as compared to the agents used alone with A549 NSLC cells.

FIG. 1B shows the IC₄₀ (ng/mL) concentrations of indibulin and erlotinib used alone and in combination, wherein the Y-scale is normalized such that each single agent IC₄₀ (ng/mL) concentration is set to 100%.

FIG. 2A shows the dose response curve of the indibulin-carboplatin combination as compared to the agents used alone with A549 NSLC cells.

FIG. 2B shows the IC₇₀ (ng/mL) concentrations of indibulin and carboplatin used alone and in combination, wherein the Y-scale is normalized such that each single agent IC₇₀ (ng/mL) concentration is set to 100%.

FIG. 3A shows the dose response curve of the indibulin-5FU combination as compared to the agents used alone with MCF7 cells.

FIG. 3B shows the IC₆₀ (ng/mL) concentrations of indibulin and 5-FU used alone and in combination, wherein the Y-scale is normalized such that each single agent IC₆₀ (ng/mL) concentration is set to 100%.

FIG. 4A shows the dose response curve of the indibulin-vinorelbine combination as compared to the agents used alone with MCF7 cells.

FIG. 4B shows the IC₆₀ (ng/mL) concentrations of indibulin and vinorelbine used alone and in combination, wherein the Y-scale is normalized such that each single agent IC₆₀ (ng/mL) concentration is set to 100%.

FIG. 5A shows the dose response curve of the indibulin-tamoxifen combination as compared to the agents used alone with MCF7 cells.

FIG. 5B shows the IC₆₀ (ng/mL) concentrations of indibulin and tamoxifen used alone and in combination, wherein the Y-scale is normalized such that each single agent IC₆₀ (ng/mL) concentration is set to 100%.

FIG. 6A shows the dose response curve of the indibulin-paclitaxel (Taxol) combination as compared to the agents used alone with MCF7 cells.

FIG. 6B shows the IC₈₄ (ng/mL) concentrations of indibulin and paclitaxel (Taxol) used alone and in combination, wherein the Y-scale is normalized such that each single agent IC₈₄ (ng/mL) concentration is set to 100%.

FIG. 7 shows that colchicine, nocodazole and podophyllotoxin compete with 3H-indibulin for tubulin binding while vinblastine and taxol do not compete.

FIG. 8 shows that indibulin inhibits about 40% of 3H-colchicine binding, nocodazole and podophyllotoxin completely inhibit 3H colchicine binding, while taxol and vinblastine have no effect.

FIG. 9 shows the effect of indibulin on polymerization of purified calf and adult bovine brain tubulin.

FIG. 10 shows that orally administered indibulin inhibits growth of MCF7 breast cancer in xenografts.

FIG. 11 shows that orally administered indibulin inhibits growth of U87 glioblastoma xenografts.

FIG. 12 shows that orally administered indibulin inhibits growth of murine renal cell carcinoma RENCA.

### DETAILED DESCRIPTION OF THE INVENTION

In certain embodiments, the invention relates to a method for the treatment of cancer, comprising administering an indolyl-3-glyoxylic acid derivative or a pharmaceutically acceptable salt thereof. In certain embodiments, the indolyl-3-glyoxylic acid derivative is indibulin. In certain embodiments, the cancer is selected from adenoid cystic carcinoma, renal cell carcinoma, breast cancer, ovarian cancer, prostate cancer, vulvar cancer, glioblastoma, and lung cancer. In certain embodiments, the cancer is selected from renal cell carcinoma, breast cancer, ovarian cancer, prostate cancer, vulvar cancer, glioblastoma, and lung cancer.

In certain embodiments, the invention relates to a method for the treatment of a cancer selected from adenoid cystic carcinoma, renal cell carcinoma, breast cancer, vulvar cancer, glioblastoma, and lung cancer comprising administering an indolyl-3-glyoxylic acid derivative, preferably indibulin. In certain embodiments, the cancer is selected from renal cell carcinoma, breast cancer, vulvar cancer, glioblastoma, and lung cancer comprising administering an indolyl-3-glyoxylic acid derivative, preferably indibulin.

One aspect of the invention relates to combination therapy, wherein an indolyl-3-glyoxylic acid derivative, such as indibulin, or a pharmaceutically acceptable salt thereof is administered with one or more other therapeutic agents and the combination shows efficacy that is greater than the efficacy of either agent being administered alone (e.g., synergistic or additive antitumor effect). Such combination treatment may be achieved by way of the simultaneous, sequential, or separate dosing of the individual components of the treatment.

In certain embodiments, an indolyl-3-glyoxylic acid derivative or a pharmaceutically acceptable salt thereof suitable for use in the methods disclosed herein is a compound disclosed in U.S. Pat. No. 6,008,231, 6,232,327, or 6,693,119, the specifications of which are hereby incorporated herein by reference in their entirety. In certain embodiments, the indolyl-3-glyoxylic acid derivative or a pharmaceutically acceptable salt thereof has a structure of Formula (I)

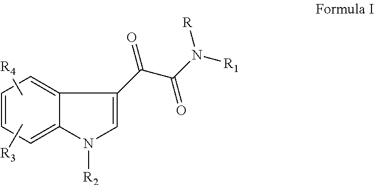

wherein

R is selected from hydrogen; (C₁-C₆)-alkyl, where the alkyl group is optionally mono- or polysubstituted with a phenyl ring which is optionally mono- or polysubstituted with halogen, (C₁-C₆)-alkyl, (C₃-C₇)-cycloalkyl, carboxyl, carboxyl esterified with C₁-C₆-alkanol, trifluoromethyl, hydroxyl, methoxy, ethoxy, benzyloxy or a benzyl group which is mono- or polysubstituted on the phenyl moiety with (C₁-C₆)-alkyl groups, halogen or trifluoromethyl; benzyloxycarbonyl; tertiary-butoxycarbonyl; and acetyl;

R₁ is selected from a phenyl ring, which is optionally mono- or polysubstituted with (C₁-C₆)-alkyl, (C₁-C₆)-alkoxy, cyano, halogen, trifluoromethyl, hydroxyl, benzyloxy, nitro, amino, (C₁-C₆)-alkylamino, (C₁-C₆)-alkoxycarbonylamino, carboxyl, or by carboxyl esterified with C₁-C₆-alkanol; a pyridine structure of the Formula (II)

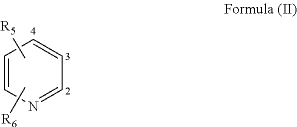

or an N-oxide thereof, where the pyridine structure is alternatively bonded to the ring carbon atoms 2, 3 or 4 and is optionally substituted with the substituents R₅ and R₆, wherein R₅ and R₆ are identical or different and are selected from (C₁-C₆)-alkyl, (C₃-C₇)-cycloalkyl, (C₁-C₆)-alkoxy, nitro, amino, hydroxyl, halogen, trifluoromethyl, ethoxycarbonylamino, and carboxyalkyloxy in which the alkyl group comprises 1-4 C atoms; 2- or 4-pyrimidinyl, wherein the 2-pyrimidinyl ring is optionally mono- or polysubstituted with a methyl group; 2-, 3-, 4- or 8-quinolyl which is optionally substituted with (C₁-C₆)-alkyl, halogen, nitro, amino or (C₁-C₆)-alkylamino; 2-, 3-, or 4-quinolylmethyl group, where the ring carbons of the pyridylmethyl radical of the quinolyl, and the quinolylmethyl are optionally substituted with (C₁-C₆)-alkyl, (C₁-C₆)-alkoxy, nitro, amino or (C₁-C₆)-alkoxycarbonylamino; and allylaminocarbonyl-2-methylprop-1-yl;

R₁, in the case in which R is hydrogen, methyl, benzyl, benzyloxycarbonyl, tert-butoxycarbonyl, or acetyl, is further selected from —CH₂COOH; —CH(CH₃)—COOH; —(CH₃)₂—CH—(CH₂)₂—CH—COO—; H₃C—H₂C—CH(CH₃)—CH(COOH)—; HO—H₂C—CH(COOH)—; phenyl-CH₂—CH(COOH)—; (4-imidazolyl)-CH₂—CH—(COOH)—; HN═(NH₂)—NH—(CH₂)₃—CH(COOH)—; H₂N—(CH₂)₄—CH(COOH)—; H₂N—CO—CH₂—CH—(COOH)—; and HOOC—(CH₂)₂—CH(COOH)—;

R₁, in the case in which R is hydrogen, benzyloxycarbonyl, tert-butoxycarbonyl, acetyl or benzyl, may be the acid radical of a natural or unnatural amino acid (e.g. α-glycyl, α-sarcosyl, α-seryl, α-phenylalanyl, α-histidyl, α-prolyl, α-arginyl, α-lysyl, α-asparagyl or α-glutamyl), where the amino groups of the respective amino acids may be protected or unprotected, wherein suitable protecting groups include, but are not limited to, benzyloxycarbonyl, tert-butoxycarbonyl, or acetyl, and in the case where R₁ is asparagyl or glutamyl, the second, unbonded carboxyl group is present as a free carboxyl group or in the form of an ester of a C₁-C₆-alkanol (e.g. as a methyl, ethyl or as a tert-butyl ester);

R and R₁ can further form, together with the nitrogen atom to which they are bonded, a piperazine ring of the Formula (III) or a homopiperazine ring, provided R₁ is an aminoalkylene group, in which

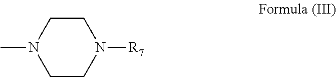

R₇ is selected from alkyl; phenyl which is optionally mono- or polysubstituted with (C₁-C₆)-alkyl, (C₁-C₆)-alkoxy, halogen, nitro, amino or by (C₁-C₆)-alkylamino; benzhydryl and bis-p-fluorobenzhydryl;

R₂ is selected from hydrogen; (C₁-C₆)-alkyl, wherein the alkyl group is optionally mono- or polysubstituted with halogen, phenyl (wherein the phenyl is optionally mono- or polysubstituted with halogen, (C₁-C₆)-alkyl, (C₃-C₇)-cycloalkyl, carboxyl, carboxyl esterified with C₁-C₆-alkanol, trifluoromethyl, hydroxyl, methoxy, ethoxy or benzyloxy), 2-quinolyl (optionally mono- or polysubstituted with halogen, (C₁-C₄)-alkyl or (C₁-C₄)-alkoxy), or 2-, 3- or 4-pyridyl (optionally mono- or polysubstituted with halogen, (C₁-C₄)-alkyl or (C₁-C₄)-alkoxy); aroyl (where the phenyl ring of the aryl moiety is optionally mono- or polysubstituted with halogen, (C₁-C₆)-alkyl, (C₃-C₇)-cycloalkyl, carboxyl, carboxyl esterified with C₁-C₆-alkanol, trifluoromethyl, hydroxyl, methoxy, ethoxy or benzyloxy);

R₃ and R₄ are identical or different and are selected from hydrogen, (C₁-C₆)-alkyl, (C₃-C₇)-cycloalkyl, (C₁-C₆)-alkanoyl, (C₁-C₆)-alkoxy, halogen, benzyloxy, nitro, amino, (C₁-C₄)-mono or dialkyl-substituted amino, (C₁-C₆)-alkoxycarbonylamino and (C₁-C₆)-alkoxycarbonylamino-(C₁-C₆)-alkyl;

Z is O or S.

In certain embodiments R₂ is selected from (C₁-C₆)-alkyl, wherein the alkyl group is optionally mono- or polysubstituted with halogen, phenyl (wherein the phenyl is optionally mono- or polysubstituted with halogen, (C₁-C₆)-alkyl, (C₃-C₇)-cycloalkyl, carboxyl, carboxyl esterified with C₁-C₆-alkanol, trifluoromethyl, hydroxyl, methoxy, ethoxy or benzyloxy), 2-quinolyl (optionally mono- or polysubstituted with halogen, (C₁-C₄)-alkyl or (C₁-C₄)-alkoxy), or 2-, 3- or 4-pyridyl (optionally mono- or polysubstituted with halogen, (C₁-C₄)-alkyl or (C₁-C₄)-alkoxy); aroyl (where the aryl moiety is optionally mono- or polysubstituted with halogen, (C₁-C₆)-alkyl, (C₃-C₇)-cycloalkyl, carboxyl, carboxyl esterified with C₁-C₆-alkanol, trifluoromethyl, hydroxyl, methoxy, ethoxy or benzyloxy).

In certain embodiments, the indolyl-3-glyoxylic acid derivative or a pharmaceutically acceptable salt thereof are of the general Formula (IV):

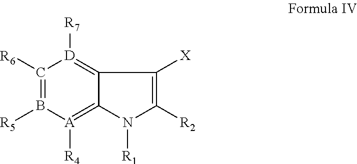

wherein:

X is hydrogen, halogen, alkyl, cycloalkyl, heterocycloalkyl, alkenyl, cycloalkenyl, heterocycloalkenyl, acyl, carboxy (—C═OOR), alkoxy, hydroxy, functionally modified hydroxy group (e.g., acyloxy) aryl, heteroaryl,

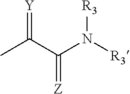

wherein Y and Z are, independently, NR, O, or S, in which R is hydrogen, alkyl, aryl, acyl, cycloalkenyl, heterocycloalkenyl, alkenyl, cycloalkenyl, heterocycloalkenyl, aminocarbonyl,

R₃ and R₃′ are, independently, alkyl, aryl, heteroaryl, or X is NR₈R₉, wherein, R₈ and R₉ are, independently, hydrogen, alkyl, cycloalkyl, heterocycloalkyl, alkenyl, cycloalkenyl, heterocycloalkenyl, acyl, aryl, or heteroaryl;

A, B, C and D are, independently, nitrogen or carbon,

provided if A is nitrogen, R₄ is absent, and if A is carbon, R₄ is either hydrogen, halogen, or alkyl;

if B is nitrogen, R₅ is absent, and if B is carbon, R₅ is hydrogen, halogen, or alkyl;

if C is nitrogen, R₆ is absent, and if C is carbon, R₆ is hydrogen, halogen, or alkyl;

if D is nitrogen, R₇ is absent, and if D is carbon, then R₇ is hydrogen, halogen, or alkyl;

R₁ is hydrogen, alkyl, aralkyl, acyl, or aryl; and

R₂ is hydrogen, alkyl, acyl, aryl, alkoxycarbonyl, aryloxycarbonyl, heteroaryloxycarbonyl, cycloalkoxycarbonyl, heterocycloalkoxycarbonyl, alkenyloxycarbonyl, cycloalkenyloxycarbonyl and heterocycloalkenyloxycarbonyl.

In certain preferred embodiments, R₁ is selected from alkyl, alkylaryl, acyl, and aryl.

In certain preferred embodiments, R₁ is a substituted benzyl group, more preferably a halogenated benzyl group (2-, 3-, or (4-halophenyl)methyl), and most preferably a (4-chlorophenyl)methyl group.

In certain preferred embodiments, R₄, R₅, R₆, and R₇ are hydrogen atoms.

In certain preferred embodiments, either R₃ or R₃′ is hydrogen and the remaining substituent (R₃ or R₃′) is a pyridinyl group (pyridine ring). More preferably, either R₃ or R₃′ is hydrogen and the remaining substituent (R₃ or R₃′) is a 4-pyridinyl group.

In certain embodiments, the indolyl-3-glyoxylic acid derivative is administered in combination with another therapeutic agent selected from erlotinib, carboplatin, 5-fluorouracil, capecitabine, paclitaxel (Taxol), tamoxifen, vinorelbine, cisplatin, gemcitabine, estramustine, doxorubicin, vinblastine, etoposide, and prednisolone. In certain embodiments, the indolyl-3-glyoxylic acid derivative, such as indibulin or a pharmaceutically acceptable salt thereof, is administered in combination with another therapeutic agent selected from erlotinib, carboplatin, 5-fluorouracil, paclitaxel, tamoxifen, and vinorelbine. In certain such embodiments, the combination is synergistic. In certain alternative embodiments, the combination is additive.

In certain embodiments, the indolyl-3-glyoxylic acid derivative is administered in combination with another therapeutic agent selected from vinca alkaloids (e.g., vinblastine, vincristine, and vinorelbine), taxanes (e.g., paclitaxel and docetaxel), epidipodophyllotoxins (e.g., etoposide, teniposide), antibiotics (e.g., dactinomycin (actinomycin D), daunorubicin, doxorubicin and idarubicin), anthracyclines, mitoxantrone, bleomycins, plicamycin (mithramycin) and mitomycin, enzymes (L-asparaginase which systemically metabolizes L-asparagine and deprives cells which do not have the capacity to synthesize their own asparagine); antiplatelet agents; antiproliferative/antimitotic alkylating agents such as nitrogen mustards (e.g., mechlorethamine, ifosphamide, cyclophosphamide and analogs, melphalan, chlorambucil), ethylenimines and methylmelamines (e.g., hexamethylmelamine and thiotepa), alkyl sulfonates (busulfan), nitrosoureas (e.g., carmustine (BCNU) and analogs, streptozocin), trazenes-dacarbazinine (DTIC); antiproliferative/antimitotic antimetabolites such as folic acid analogs (e.g., methotrexate), pyrimidine analogs (e.g., fluorouracil, floxuridine, and cytarabine), purine analogs and related inhibitors (e.g., mercaptopurine, thioguanine, pentostatin and 2-chlorodeoxyadenosine); aromatase inhibitors (e.g., anastrozole, exemestane, and letrozole); and platinum coordination complexes (e.g., cisplatin, carboplatin), procarbazine, hydroxyurea, mitotane, aminoglutethimide; hormones (e.g., estrogen) and hormone agonists such as leutinizing hormone releasing hormone (LHRH) agonists (e.g., goserelin, leuprolide and triptorelin). Other chemotherapeutic agents may include mechlorethamine, camptothecin, ifosfamide, tamoxifen, raloxifene, gemcitabine, navelbine, or any analog or derivative variant of the foregoing.

In certain embodiments, the indolyl-3-glyoxylic acid derivative is administered in combination with another therapeutic agent selected from a tubulin binding agent, a kinase inhibitor (e.g., a receptor tyrosine kinase inhibitor), an anti-metabolic agent, a DNA synthesis inhibitor, and a DNA damaging agent.

One aspect of the invention provides combination therapy, wherein an indolyl-3-glyoxylic acid derivative, such as indibulin or a pharmaceutically acceptable salt thereof, is administered with one or more other therapeutic agents and the combination is beneficial to efficacy, optionally additive or synergistic. Such combination treatment may be achieved by way of the simultaneous, sequential, or separate dosing of the individual components of the treatment.

In certain embodiments, an indolyl-3-glyoxylic acid derivative, such as indibulin is administered in combination with another therapeutic agent selected from erlotinib, carboplatin, 5-fluorouracil, capecitabine, paclitaxel, tamoxifen, vinorelbine, cisplatin, gemcitabine, estramustine, doxorubicin, vinblastine, etoposide, and prednisolone. In certain embodiments, indibulin is administered in combination with another therapeutic agent selected from erlotinib, carboplatin, 5-fluorouracil, paclitaxel (Taxol), tamoxifen, and vinorelbine. In certain such embodiments, the combination is synergistic. In certain alternative embodiments, the combination is additive.

In certain embodiments, indibulin is administered in combination with another therapeutic agent selected from vinca alkaloids (e.g., vinblastine, vincristine, and vinorelbine), taxanes (e.g., paclitaxel and docetaxel), epidipodophyllotoxins (e.g., etoposide, teniposide), antibiotics (e.g., dactinomycin (actinomycin D), daunorubicin, doxorubicin and idarubicin), anthracyclines, mitoxantrone, bleomycins, plicamycin (mithramycin) and mitomycin, enzymes (L-asparaginase which systemically metabolizes L-asparagine and deprives cells which do not have the capacity to synthesize their own asparagine); antiplatelet agents; antiproliferative/antimitotic alkylating agents such as nitrogen mustards (e.g., mechlorethamine, ifosphamide, cyclophosphamide and analogs, melphalan, chlorambucil), ethylenimines and methylmelamines (e.g., hexamethylmelamine and thiotepa), alkyl sulfonates (busulfan), nitrosoureas (e.g., carmustine (BCNU) and analogs, streptozocin), trazenes—dacarbazinine (DTIC); antiproliferative/antimitotic antimetabolites such as folic acid analogs (e.g., methotrexate), pyrimidine analogs (e.g., fluorouracil, floxuridine, and cytarabine), purine analogs and related inhibitors (e.g., mercaptopurine, thioguanine, pentostatin and 2-chlorodeoxyadenosine); aromatase inhibitors (e.g., anastrozole, exemestane, and letrozole); and platinum coordination complexes (e.g., cisplatin, carboplatin), procarbazine, hydroxyurea, mitotane, aminoglutethimide; hormones (e.g., estrogen) and hormone agonists such as leutinizing hormone releasing hormone (LHRH) agonists (e.g., goserelin, leuprolide and triptorelin). Other chemotherapeutic agents may include mechlorethamine, camptothecin, ifosfamide, tamoxifen, raloxifene, gemcitabine, navelbine, or any analog or derivative variant of the foregoing.

In certain embodiments, indibulin is administered in combination with another therapeutic agent selected from a tubulin binding agent, a kinase inhibitor (e.g., a receptor tyrosine kinase inhibitor), an anti-metabolic agent, a DNA synthesis inhibitor, and a DNA damaging agent.

Another aspect of the invention relates to methods for the treatment of cancer, comprising administering an indolyl-3-glyoxylic acid derivative or a pharmaceutically acceptable salt thereof, in combination with another therapeutic agent, wherein the combination is beneficial to efficacy, optionally additive or synergistic. In certain such embodiments, the invention relates to methods for the treatment of a cancer selected from lung, breast, ovarian, and prostate cancer. In certain embodiments, the invention relates to methods for the treatment of a cancer selected from lung and breast cancer. In certain embodiments, the indolyl-3-glyoxylic acid derivative or a pharmaceutically acceptable salt thereof has a structure of Formula (I).

Another aspect of the invention relates to methods for the treatment of cancer, comprising administering indibulin or a pharmaceutically acceptable salt thereof, in combination with another therapeutic agent, wherein the combination is beneficial to efficacy, optionally additive or synergistic. In certain such embodiments, the invention relates to methods for the treatment of a cancer selected from lung, breast, ovarian, and prostate cancer. In certain embodiments, the invention relates to methods for the treatment of a cancer selected from lung and breast cancer.

As used herein, and as well understood in the art, “treatment” is an approach for obtaining beneficial or desired results, including clinical results. Beneficial or desired clinical results can include, but are not limited to, alleviation or amelioration of one or more symptoms or conditions, diminishment of extent of disease, stabilized (i.e., not worsening) state of disease, preventing spread of disease, delay or slowing of disease progression, amelioration or palliation of the disease state, and remission (whether partial or total), whether detectable or undetectable. “Treatment” can also mean prolonging survival as compared to expected survival in the absence of receiving treatment, or as compared to a control patient or patients not receiving treatment.

Another aspect of the invention relates to a kit, comprising an indolyl-3-glyoxylic acid derivative or a pharmaceutically acceptable salt thereof and another therapeutic agent. In certain embodiments, the other therapeutic agent is selected from erlotinib, carboplatin, 5-fluorouracil, capecitabine, paclitaxel, tamoxifen, vinorelbine, cisplatin, gemcitabine, estramustine, doxorubicin, vinblastine, etoposide, and prednisolone. In certain embodiments, the other therapeutic agent is selected from erlotinib, carboplatin, 5-fluorouracil, paclitaxel, tamoxifen, and vinorelbine. In certain embodiments, the indolyl-3-glyoxylic acid derivative or a pharmaceutically acceptable salt thereof has a structure of Formula (I).

Another aspect of the invention relates to a kit, comprising indibulin or a pharmaceutically acceptable salt thereof and another therapeutic agent. In certain embodiments, the other therapeutic agent is selected from erlotinib, carboplatin, 5-fluorouracil, capecitabine, paclitaxel, tamoxifen, vinorelbine, cisplatin, gemcitabine, estramustine, doxorubicin, vinblastine, etoposide, and prednisolone. In certain embodiments, the other therapeutic agent is selected from erlotinib, carboplatin, 5-fluorouracil, paclitaxel, tamoxifen, and vinorelbine.

Administration of the indolyl-3-glyoxylic acid derivative may precede or follow the other therapeutic agent by intervals ranging from minutes to days. In certain such embodiments, the indolyl-3-glyoxylic acid derivative and the other therapeutic agent may be administered within about 1 minute, about 5 minutes, about 10 minutes, about 30 minutes, about 60 minutes, about 2 hours, about 4 hours, about 6 hours, 8 hours, about 10 hours, about 12 hours, about 18 hours, about 24 hours, about 36 hours, or even about 48 hours or more of one another. Preferably, administration of the indolyl-3-glyoxylic acid derivative and the other therapeutic agent will be within about 1 minute, about 5 minutes, about 30 minutes, or even about 60 minutes of one another.

In certain embodiments, the indolyl-3-glyoxylic acid derivative and the other therapeutic agent may be administered according to different dosing schedules (e.g., indibulin, for example may be administered once a day while the other therapeutic agent may be administered only once every three weeks) such that in some instances administration of the indolyl-3-glyoxylic acid derivative and the other therapeutic agent will be within about 60 minutes of one another, while in other instances, administration of the indolyl-3-glyoxylic acid derivative and the other therapeutic agent will be within days or even weeks of one another.

As used herein, the term “regimen” is a predetermined schedule of one or more therapeutic agents for the treatment of a cancer. Accordingly, when a therapeutic agent is administered “alone,” the regimen does not include the use of another therapeutic agent for the treatment of cancer.

In certain embodiments, the indolyl-3-glyoxylic acid derivative, such as indibulin, is administered once daily for fourteen days every three weeks. In certain embodiments, such dosing is by oral administration of, for example, a liquid or capsule.

In certain embodiments where the indolyl-3-glyoxylic acid derivative is administered as a liquid, a single dose comprises from about 20 to about 80 mg. In certain embodiments where the indolyl-3-glyoxylic acid derivative is administered as an oral dosage form, a dose is about 100 to about 250 mg, or even more. In certain embodiments a daily dose may be about 100 to 2000 mg, about 250 to about 2000 mg, or even about 500 to about 2000 mg. In certain embodiments a daily dose may be greater than or equal to 500 mg, 900 mg, 1000 mg, 1200 mg, 1500 mg, 1800 mg, 2000 mg, or even 2500 mg.

In certain embodiments, the indolyl-3-glyoxylic acid derivative, such as indibulin may be administered, e.g., as an oral dosage form (e.g., as described in the preceding paragraph), every two days, every three days, every other day, daily, twice daily, or even three times daily, or in any other regular regimen, e.g. continuous treatment. Such administration may be for a duration of three weeks, four weeks, five weeks or more, such as six months, one year, two years, or more.

As used herein the term “continuous treatment” means a dose in which the patient goes no more than one or at most two consecutive days without a dose for as long as there is a perceived benefit, e.g. such as six months, one year, two years, five years, or even more. In certain embodiments the indolyl-3-glyoxylic acid derivative, such as indibulin is administered twice daily as an oral dosage form.

In certain embodiments, the indolyl-3-glyoxylic acid derivative, such as indibulin, is administered as an oral dosage form, preferably twice daily. In certain embodiments, the indolyl-3-glyoxylic acid derivative is administered as a continuous treatment, preferably as an oral dosage form. In certain such embodiments, the continuous treatment comprises administration of an oral dosage form twice daily. Suitable oral dosage forms include, but are not limited to, capsules, such as hard gelatin capsules. In certain such embodiments each dose comprises about 400 mg of the indolyl-3-glyoxylic acid derivative, such as indibulin, administered twice daily.

In certain embodiments, the indolyl-3-glyoxylic acid derivative, such as indibulin is administered with food. In certain such embodiments administration with food may increase bioavailability.

In certain embodiments, combinations as described herein may be synergistic in nature, meaning that the therapeutic effect of the combination of the indolyl-3-glyoxylic acid derivative and the other therapeutic agent(s) is greater than the sum of the individual effects.

In certain embodiments, combinations as described herein may be additive in nature, meaning that the therapeutic effect of the combination of the indolyl-3-glyoxylic acid derivative and the other therapeutic agent(s) is greater than the effect of each agent individually (i.e., the therapeutic effect is the sum of the individual effects).

Compounds described herein can be administered in various forms, depending on the disorder to be treated and the age, condition, and body weight of the patient, as is well known in the art. For example, where the compounds are to be administered orally, they may be formulated as tablets, capsules, granules, powders, or syrups; or for parenteral administration, they may be formulated as injections (intravenous, intramuscular, or subcutaneous), or drop infusion preparations. These formulations can be prepared by conventional means, and if desired, the active ingredient may be mixed with any conventional additive or excipient, such as a binder, a disintegrating agent, a lubricant, a corrigent, a solubilizing agent, a suspension aid, an emulsifying agent, a coating agent, a cyclodextrin, and/or a buffer. The dosage will vary depending on the symptoms, age and body weight of the patient, the nature and severity of the disorder to be treated or prevented, the route of administration and the form of the drug. The amount of active ingredient which can be combined with a carrier material to produce a single dosage form will generally be that amount of the compound which produces a therapeutic effect.

In certain embodiments, the compounds described herein may be administered as a particulate composition, preferably an aqueous suspension of nanoparticles with at least one surfactant selected from ionic surfactants, non-ionic surfactants, zwitterionic surfactants, biologically derived surfactants, amino acids and their derivatives and combinations thereof. Such particulate compositions may be administered in any suitable way, including, but not limited to orally or parenterally. In certain embodiments, one or more compounds described herein are present in the composition in an amount from about 0.01% to about 20% (w/v), preferably from about 0.055 to about 15% w/v, or even from about 0.1% to about 10% w/v. In certain embodiments, the particles will vary in size from about 15 nm to 50 microns, preferably from about 50 nm to 10 microns, or even from about 50 nm to 2 microns. When the particles are prepared for administration by injection, it is preferred that the have a particle size of less than about 5 microns (microparticles) or even less than about 2 microns (nanoparticles). Particulate compositions described herein are also described in WO 2006/052712, the disclosure of which is incorporated herein in its entirety.

Suitable surfactants for coating the particles in the present invention can be selected from ionic surfactants, nonionic surfactants, zwitterionic surfactants, phospholipids, biologically derived surfactants or amino acids and their derivatives. Ionic surfactants can be anionic or cationic. The surfactants are present in the compositions in an amount of from about 0.01% to 10% w/v, and preferably from about 0.05% to about 5% w/v.

Suitable anionic surfactants include but are not limited to: alkyl sulfonates, aryl sulfonates, alkyl phosphates, alkyl phosphonates, potassium laurate, sodium lauryl sulfate, sodium dodecylsulfate, alkyl polyoxyethylene sulfates, sodium alginate, phosphatidic acid and their salts, sodium carboxymethylcellulose, bile acids and their salts (e.g., salts of cholic acid, deoxycholic acid, glycocholic acid, taurocholic acid, and glycodeoxycholic acid), and calcium carboxymethylcellulose, stearic acid and its salts (e.g., sodium and calcium stearate), phosphates, sodium dodecylsulfate, carboxymethylcellulose calcium, carboxymethylcellulose sodium, dioctyl sodium sulfosuccinate (DOSS), dialkylesters of sodium sulfosuccinic acid, sodium lauryl sulfate and phospholipids.

Suitable cationic surfactants include but are not limited to: quaternary ammonium compounds, benzalkonium chloride, cetyltrimethylammonium bromide, chitosans, lauryldimethylbenzylammonium chloride, acyl carnitine hydrochlorides, alkyl pyridinium halides, cetyl pyridinium chloride, cationic lipids, polymethylmethacrylate trimethylammonium bromide, sulfonium compounds, polyvinylpyrrolidone-2-dimethylaminoethyl methacrylate dimethyl sulfate, hexadecyltrimethyl ammonium bromide, phosphonium compounds, quaternary ammonium compounds, benzyl-di(2-chloroethyl)ethylammonium bromide, coconut trimethyl ammonium chloride, coconut trimethyl ammonium bromide, coconut methyl dihydroxyethyl ammonium chloride, coconut methyl dihydroxyethyl ammonium bromide, decyl triethyl ammonium chloride, decyl dimethyl hydroxyethyl ammonium chloride, decyl dimethyl hydroxyethyl ammonium chloride bromide, C₁₂₋₁₅-dimethyl hydroxyethyl ammonium chloride, C₁₂₋₁₅-dimethyl hydroxyethyl ammonium chloride bromide, coconut dimethyl hydroxyethyl ammonium chloride, coconut dimethyl hydroxyethyl ammonium bromide, myristyl trimethyl ammonium methyl sulfate, lauryl dimethyl benzyl ammonium chloride, lauryl dimethyl benzyl ammonium bromide, lauryl dimethyl (ethenoxy)₄ ammonium chloride, lauryl dimethyl (ethenoxy)₄ ammonium bromide, N-alkyl(C₁₂₋₁₈)dimethylbenzyl ammonium chloride, N-alkyl(C₁₄₋₁₈)dimethyl-benzyl ammonium chloride, N-tetradecylidmethylbenzyl ammonium chloride monohydrate, dimethyl didecyl ammonium chloride, N-alkyl and (C₁₂₋₁₄)dimethyl-1-napthylmethyl ammonium chloride, trimethylammonium halide alkyl-trimethylammonium salts, dialkyl-dimethylammonium salts, lauryl trimethyl ammonium chloride, ethoxylated alkyamidoalkyldialkylammonium salts, ethoxylated trialkyl ammonium salts; dialkylbenzene dialkylammonium chloride, N-didecyldimethyl ammonium chloride, N-tetradecyldimethylbenzyl ammonium chloride monohydrate, N-alkyl(C₁₂₋₁₄₎dimethyl 1-naphthylmethyl ammonium chloride, dodecyldimethylbenzyl ammonium chloride, dialkyl benzenealkyl ammonium chloride, lauryl trimethyl ammonium chloride, alkylbenzyl methyl ammonium chloride, alkyl benzyl dimethyl ammonium bromide, C₁₂ trimethyl ammonium bromides, C₁₅trimethyl ammonium bromides, C₁₇trimethyl ammonium bromides, dodecylbenzyl triethyl ammonium chloride, poly-diallyldimethylammonium chloride (DADMAC), dimethyl ammonium chlorides, alkyldimethylammonium halogenides, tricetyl methyl ammonium chloride, decyltrimethylammonium bromide, dodecyltriethylammonium bromide, tetradecyltrimethylammonium bromide, methyl trioctylammonium chloride, “POLYQUAT 10” (a mixture of polymeric quarternary ammonium compounds), tetrabutylammonium bromide, benzyl trimethylammonium bromide, choline esters, benzalkonium chloride, stearalkonium chloride, cetyl pyridinium bromide, cetyl pyridinium chloride, halide salts of quaternized polyoxyethylalkylamines, alkyl pyridinium salts, amines, amine salts, imide azolinium salts, protonated quaternary acrylamides, methylated quaternary polymers, cationic guar gum, benzalkonium chloride, dodecyl trimethyl ammonium bromide, triethanolamine, and poloxamines.

Suitable nonionic surfactants include but are not limited to: polyoxyethylene fatty alcohol ethers, polyoxyethylene sorbitan fatty acid esters, polyoxyethylene fatty acid esters, sorbitan esters, glyceryl esters, glycerol monostearate, polyethylene glycols, polypropylene glycols, polypropylene glycol esters, cetyl alcohol, cetostearyl alcohol, stearyl alcohol, aryl alkyl polyether alcohols, polyoxyethylene-polyoxypropylene copolymers, poloxamers, poloxamines, methylcellulose, hydroxycellulose, hydroxymethylcellulose, hydroxypropylcellulose, hydroxypropylmethylcellulose, noncrystalline cellulose, polysaccharides, starch, starch derivatives, hydroxyethylstarch, polyvinyl alcohol, polyvinylpyrrolidone, triethanolamine stearate, amine oxides, dextran, glycerol, gum acacia, cholesterol, tragacanth, glycerol monostearate, cetostearyl alcohol, cetomacrogol emulsifying wax, sorbitan esters, polyoxyethylene alkyl ethers, polyoxyethylene castor oil derivatives, polyoxyethylene sorbitan fatty acid esters, polyethylene glycols, polyoxyethylene stearates, hydroxypropyl celluloses, hydroxypropyl methylcellulose, methylcellulose, hydroxyethylcellulose, hydroxypropylmethylcellulose phthalate, noncrystalline cellulose, polyvinyl alcohol, polyvinylpyrrolidone, 4-(1,1,3,3-tetramethylbutyl)phenol polymer with ethylene oxide and formaldehyde, poloxamers, alkyl aryl polyether sulfonates, mixtures of sucrose stearate and sucrose distearate, C₁₈H₃₇CH₂C(O)N(CH₃)CH₂(CHOH)₄(CH₂OH)₂, p-isononylphenoxypoly(glycidol), decanoyl-N-methylglucamide, n-decyl-β-D-glucopyranoside, n-decyl-β-D-maltopyranoside, n-dodecyl-β-D-glucopyranoside, n-dodecyl-β-D-maltoside, heptanoyl-N-methylglucamide, n-heptyl-β-D-glucopy-ranoside, n-heptyl-β-D-thioglucoside, n-hexyl-β-D-glucopyranoside; nonanoyl-N-methylglucamide, n-nonyl-β-D-glucopyranoside, octanoyl-N-methylglucamide, n-octyl-β-D-glucopyranoside, octyl-β-D-thioglucopyranoside, PEG-cholesterol, PEG-cholesterol derivatives, PEG-vitamin A, PEG-vitamin E, and random copolymers of vinyl acetate and vinyl pyrrolidone.

Zwitterionic surfactants are electrically neutral but possess local positive and negative charges within the same molecule. The net charge on the molecule may depend on the pH, and therefore at low pH some zwitterionic surfactants may act as cationic surfactants while at high pH they may also act an anionic surfactants. Suitable zwitterionic surfactants include but are not limited to zwitterionic phospholipids. These phospholipids include phosphatidylcholine, phosphatidylethanolamine, diacyl-glycero-phosphoethanolamine (such as dimyristoylglycero-phosphoethanolamine (DMPE), dipalmitoyl-glycero-phosphoethanolamine (DPPE), distearoyl-glycero-phosphoethanolamine (DSPE), and dioleolyl-glycerophosphoethanolamine (DOPE), pegylated phospholipids, PEG-phosphatidylcholine, PEG-diacyl-glycero-phosphoethanolamine, PEG-phosphatidylethanolamine, PEG-diacyl-glycerophosphoethanolamine, PEG-dimyristoyl-glycero-phosphoethanolamine, PEG-dipalmitoylglycero-phosphoethanolamine, PEG-distearoyl-glycero-phosphoethanolamine, PEG-dioleolyl-glycero-phosphoethanolamine, methoxy polyethylene glycol (mPEG)-phospholipids, mPEG-phosphatidylcholine, mPEG-diacyl-glycero-phosphoethanolamine, mPEG-phosphatidylethanolamine, mPEG-diacyl-glycero-phosphoethanolamine, mPEG-dimyristoyl-glycero-phosphoethanolamine, mPEG-dipalmitoyl-glycerophosphoethanolamine, mPEG-distearoyl-glycero-phosphoethanolamine, and mPEG-dioleolylglycero-phosphoethanolamine.

Mixtures of phospholipids that include anionic and zwitterionic phospholipids may be employed in this invention. Such mixtures include but are not limited to lysophospholipids, egg or soybean phospholipid or any combination thereof.

Suitable biologically derived surfactants include, but are not limited to: lipoproteins, gelatin, casein, lysozyme, albumin, casein, heparin, hirudin, or other proteins.

A preferred ionic surfactant is a bile salt, and a preferred bile salt is sodium deoxycholate. A preferred nonionic surfactant is a polyalkoxyether, and preferred polyalkoxyethers are polyoxyethylene-polyoxypropylene triblock copolymers such as Poloxamer 188 and Poloxamer 407. Another preferred surfactant is a lipid in which a polyalkoxyether is covalently attached to a lipid through an ether linkage. A preferred surfactant of this class is a pegylated phospholipid. Another preferred surfactant is a pegylated phospholipid methyl ether (for example, mPEG-DSPE).

In a preferred embodiment of the present invention, the particles are suspended in an aqueous medium further including a pH adjusting agent. Suitable pH adjusting agents include, but are not limited to, sodium hydroxide, hydrochloric acid, tris buffer, mono-, di-tricarboxylic acids and their salts, citrate buffer, phosphate, glycerol-1-phosphate, glycercol-2-phosphate, acetate, lactate, tris(hydroxymethyl)aminomethane, aminosaccharides, mono-, di- and trialkylated amines, meglumine (N-methylglucosamine), and amino acids. The aqueous medium may additionally include an osmotic pressure adjusting agent, such as but not limited to glycerin, a monosaccharide such as dextrose, a disaccharide such as sucrose, trehalose and maltose, a trisaccharide such as raffinose, and sugar alcohols such as mannitol and sorbitol.

In an embodiment of the present invention, the aqueous medium of the particle suspension composition is removed to form dry particles. The method to remove the aqueous medium can be any method known in the art. One example is evaporation. Another example is freeze-drying or lyophilization. The dry particles may then be formulated into any acceptable physical form including, but not limited to, solutions, tablets, capsules, suspensions, creams, lotions, emulsions, aerosols, powders, incorporation into reservoir or matrix devices for sustained release (such as implants or transdermal patches), and the like. The aqueous suspension of the present invention may also be frozen to improve stability upon storage. Freezing of an aqueous suspension to improve stability is disclosed in the commonly assigned and co-pending U.S. patent application Ser. No. 10/270,267, which is incorporated herein by reference in its entirety.

Preferred compositions comprise an aqueous suspension of particles of tubulin inhibitor present at 0.05% to 10% w/v, the particles are coated with 0.05% to 5% w/v of an ionic surfactant (e.g., deoxycholate) or a zwitterionic surfactant (e.g., mPEG-DSPE), and 0.05% to 5% w/v polyalkoxyether (for example, Poloxamer 188), and glycerin added to adjust osmotic pressure of the formulation.

The particle suspensions of the present invention can be prepared by methods known to those skilled in the art and those methods described below.

In certain embodiments, compounds as disclosed herein may be administered as a pharmaceutical formulation for oral administration, wherein the formulation comprises a granulate containing micronized indolyl-3-glyoxylic acid derivative, such as indibulin, having a particle size of less than 20 μm for at least 99 vol.-% of the particles, at least one hydrophilic surfactant, and one or more additional capsulation excipients. Such pharmaceutical formulations are described in WO 2006/133835 which is incorporated herein by reference in its entirety.

In certain preferred embodiment, the micronized indolyl-3-glyoxylic acid derivative has a particle size of less than 10 μm for at least 90 vol. % of the particles, less than 10 μm for at least 99 vol. % of the particles. In certain preferred embodiments, the micronized indolyl-3-glyoxylic acid derivative has a mean particle size in the range of 2 to 4 μm.

In a preferred embodiment of the present invention, the pharmaceutical formulation comprises a indolyl-3-glyoxylic acid derivative in an amount of about 10 to about 50 weight %, the at least one hydrophilic surfactant in an amount of about 1 to about 10 weight %, and one or more capsulation excipients in an amount of about 40 to about 80 weight %, the three constituents always adding up to 100 weight % of said pharmaceutical formulation.

The hydrophilic surfactant is not subject to any particular limitation as long as it is capable of acting as an oil-in-water surfactant. Preferably, the one or more hydrophilic surfactant(s) is/are selected from the group consisting of polysorbates, poloxamers, cremophors and polyalkylene glycols. Any type of polysorbate can be employed, but particularly the polysorbate is selected from polysorbate 20, polysorbate 40, polysorbate 60 or polysorbate 80, more preferred from polysorbate 80. Further, any type of poloxamers can be employed. Poloxamers are surfactant-like block polymers having a central polypropylene glycol moiety which on both terminal ends is connected to a macrogol moiety. Typical poloxamers suited for the present invention are poloxamers 188 and 407, particularly poloxamer 188. Cremophors are non-ionic emulsifiers obtained by causing ethylene oxide to react with castor oil particularly in a molar ratio of about 35 moles to 1 mole. Other common names are polyoxyethyleneglyceroltriricinoleate 35 or polyoxyl 35 castor oil. A typical cremophor is for example Cremophor® EL supplied by BASF AG, Germany.

As capsulation excipients those which are common in the art can be suitably used in the present invention. In particular, those capsulation excipients can comprise cellulose such as microcrystalline cellulose or a derivative thereof, gelatin, starch, particularly corn starch, and highly disperse silicon dioxide (aerosil). Typically, the capsulation excipients comprise a mixture of microcrystalline cellulose, gelatin, corn starch and aerosil. For example, corn starch and microcrystalline can serve as a filling mass and degradants. Highly disperse silicon dioxide (aerosil) acts in turn to make the mass fluent. Gelatin usually serves as an adhesive to get homogeneous granules.

In a preferred embodiment of the present invention the granules constituting said pharmaceutical formulation are covered by an outer phase composed of a mixture comprising starch, particularly corn starch, highly dispersed silicon dioxide and magnesium stearate. Such an outer phase properly enables the encapsulation the granules.

In certain embodiments, the pharmaceutical formulation is a tablet prepared using said pharmaceutical formulation or a capsule filled with said pharmaceutical formulation, respectively.

The pharmaceutical formulation of the indolyl-3-glyoxylic acid derivative, such as indibulin, may be based on micronization of the compound combined with a granulation procedure using a hydrophilic surfactant (e.g., polysorbate, poloxamer, cremophor) and common capsulation excipients (e.g., cellulose, starch, highly disperse silicon dioxide, etc).

In certain embodiments, the indolyl-3-glyoxylic acid derivative and the other therapeutic agent may be in the same form (e.g., both may be administered as tablets or both may be administered intravenously) while in certain alternative embodiments, the indolyl-3-glyoxylic acid derivative and the other therapeutic agent may be in different forms (e.g. one may be administered as a tablet while the other is administered intravenously).

The precise time of administration and/or amount of the composition that will yield the most effective results in terms of efficacy of treatment in a given patient will depend upon the activity, pharmacokinetics, and bioavailability of a particular compound, physiological condition of the patient (including age, sex, disease type and stage, general physical condition, responsiveness to a given dosage, and type of medication), route of administration, etc. However, the above guidelines can be used as the basis for fine-tuning the treatment, e.g., determining the optimum time and/or amount of administration, which will require no more than routine experimentation consisting of monitoring the subject and adjusting the dosage and/or timing.

The phrase “pharmaceutically acceptable” is employed herein to refer to those ligands, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable carrier” as used herein means a pharmaceutically acceptable material, composition, or vehicle, such as a liquid or solid filler, diluent, excipient, solvent or encapsulating material. Each carrier must be “acceptable” in the sense of being compatible with the other ingredients of the formulation and not injurious to the patient. Some examples of materials which can serve as pharmaceutically acceptable carriers include: (1) sugars, such as lactose, glucose, and sucrose; (2) starches, such as corn starch, potato starch, and substituted or unsubstituted β-cyclodextrin; (3) cellulose, and its derivatives, such as sodium carboxymethyl cellulose, ethyl cellulose, and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil, and soybean oil; (10) glycols, such as propylene glycol; (11) polyols, such as glycerin, sorbitol, mannitol, and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations. In certain embodiments, pharmaceutical compositions of the present invention are non-pyrogenic, i.e., do not induce significant temperature elevations when administered to a patient.

The term “pharmaceutically acceptable salt” refers to the relatively non-toxic, inorganic and organic acid addition salts of the inhibitor(s). These salts can be prepared in situ during the final isolation and purification of the inhibitor(s), or by separately reacting a purified inhibitor(s) in its free base form with a suitable organic or inorganic acid, and isolating the salt thus formed. Representative salts include the hydrobromide, hydrochloride, sulfate, bisulfate, phosphate, nitrate, acetate, trifluoroacetate, citrate, embonate, methanesulfonate, valerate, oleate, palmitate, stearate, laurate, benzoate, lactate, phosphate, succinate, tosylate, citrate, malonate, maleate, fumarate, succinate, tartrate, naphthylate, mesylate, gluconate, glucouronate, glucoheptonate, 2-hydroxyethansulfonate, lactobionate, laurylsulphonate salts, and amino acid salts, and the like. (See, for example, Berge et al. (1977) “Pharmaceutical Salts”, J. Pharm. Sci. 66: 1-19.)

In other cases, the inhibitors useful in the methods of the present invention may contain one or more acidic functional groups and, thus, are capable of forming pharmaceutically acceptable salts with pharmaceutically acceptable bases. The term “pharmaceutically acceptable salts” in these instances refers to the relatively non-toxic inorganic and organic base addition salts of an inhibitor(s). These salts can likewise be prepared in situ during the final isolation and purification of the inhibitor(s), or by separately reacting the purified inhibitor(s) in its free acid form with a suitable base, such as the hydroxide, carbonate, or bicarbonate of a pharmaceutically acceptable metal cation, with ammonia, or with a pharmaceutically acceptable organic primary, secondary, or tertiary amine. Representative alkali or alkaline earth salts include the lithium, sodium, potassium, calcium, magnesium, and aluminum salts, and the like. Representative organic amines useful for the formation of base addition salts include ethylamine, diethylamine, ethylenediamine, ethanolamine, diethanolamine, piperazine, and the like (see, for example, Berge et al., supra).

Wetting agents, emulsifiers, and lubricants, such as sodium lauryl sulfate and magnesium stearate, as well as coloring agents, release agents, coating agents, sweetening, flavoring, and perfuming agents, preservatives and antioxidants can also be present in the compositions.

Examples of pharmaceutically acceptable antioxidants include: (1) water soluble antioxidants, such as ascorbic acid, cysteine hydrochloride, sodium bisulfate, sodium metabisulfite, sodium sulfite, and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

Formulations suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert matrix, such as gelatin and glycerin, or sucrose and acacia) and/or as mouthwashes, and the like, each containing a predetermined amount of an inhibitor(s) as an active ingredient. A composition may also be administered as a bolus, electuary, or paste.

In solid dosage forms for oral administration (capsules, tablets, pills, dragees, powders, granules, and the like), the active ingredient is mixed with one or more pharmaceutically acceptable carriers, such as sodium citrate or dicalcium phosphate, and/or any of the following: (1) fillers or extenders, such as starches, cyclodextrins, lactose, sucrose, glucose, mannitol, and/or silicic acid; (2) binders, such as, for example, carboxymethylcellulose, alginates, gelatin, polyvinyl pyrrolidone, sucrose, and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, acetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; and (10) coloring agents. In the case of capsules, tablets, and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols, and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered inhibitor(s) moistened with an inert liquid diluent.

Tablets, and other solid dosage forms, such as dragees, capsules, pills, and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes, and/or microspheres. They may be sterilized by, for example, filtration through a bacteria-retaining filter, or by incorporating sterilizing agents in the form of sterile solid compositions which can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions which can be used include polymeric substances and waxes. The active ingredient can also be in micro-encapsulated form, if appropriate, with one or more of the above-described excipients.

Liquid dosage forms for oral administration include pharmaceutically acceptable emulsions, microemulsions, solutions, suspensions, syrups, and elixirs. In addition to the active ingredient, the liquid dosage forms may contain inert diluents commonly used in the art, such as, for example, water or other solvents, solubilizing agents, and emulsifiers such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor, and sesame oils), glycerol, tetrahydrofuryl alcohol, polyethylene glycols, and fatty acid esters of sorbitan, and mixtures thereof.

Besides inert diluents, the oral compositions can also include adjuvants such as wetting agents, emulsifying and suspending agents, sweetening, flavoring, coloring, perfuming, and preservative agents.

Suspensions, in addition to the active inhibitor(s) may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Pharmaceutical compositions of this invention suitable for parenteral administration comprise one or more inhibitors(s) in combination with one or more pharmaceutically acceptable sterile aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and nonaqueous carriers which may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents, and dispersing agents. Prevention of the action of microorganisms may be ensured by the inclusion of various antibacterial and antifungal agents, for example, paraben, chlorobutanol, phenol sorbic acid, and the like. It may also be desirable to include tonicity-adjusting agents, such as sugars, sodium chloride, and the like into the compositions. In addition, prolonged absorption of the injectable pharmaceutical form may be brought about by the inclusion of agents which delay absorption such as aluminum monostearate and gelatin.

In some cases, in order to prolong the effect of a drug, it is desirable to slow the absorption of the drug from subcutaneous or intramuscular injection. For example, delayed absorption of a parenterally administered drug form is accomplished by dissolving or suspending the drug in an oil vehicle.

Injectable depot forms are made by forming microencapsule matrices of inhibitor(s) in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions which are compatible with body tissue.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection, and infusion.

The phrases “systemic administration,” “administered systemically,” “peripheral administration” and “administered peripherally” as used herein mean the administration of a ligand, drug, or other material other than directly into the central nervous system, such that it enters the patient's system and thus, is subject to metabolism and other like processes, for example, subcutaneous administration.

Administration of the therapeutic compositions of the present invention to a patient will follow general protocols for the administration of chemotherapeutics, taking into account the toxicity, if any. It is expected that the treatment cycles would be repeated as necessary. It also is contemplated that various standard therapies or adjunct cancer therapies, as well as surgical intervention, may be applied in combination with the described arsenical agent.

Regardless of the route of administration selected, the inhibitor(s), which may be used in a suitable hydrated form, and/or the pharmaceutical compositions of the present invention, are formulated into pharmaceutically acceptable dosage forms by conventional methods known to those of skill in the art.

Actual dosage levels of the active ingredients in the pharmaceutical compositions of this invention may be varied so as to obtain an amount of the active ingredient which is effective to achieve the desired therapeutic response for a particular patient, composition, and mode of administration, without being toxic to the patient.

The following examples are presented in order to more fully illustrate the preferred embodiments of the invention. These examples should in no way be construed as limiting the scope of the invention, as defined by the appended claims.

### EXAMPLES

### Indibulin Combinations

The monolayer assay determines the in vitro antitumor activity of test compounds and their capacity to inhibit survival and proliferation of established tumor cell lines. These effects can be determined by establishing the number of viable cells based on measuring the DNA content using propidium iodide. In a first step, indibulin and the other agents were tested separately each at ten different concentrations for their antitumor activity in cell lines MCF-7 (breast), A549 (NSCLC), SKOV3 (ovarian), and PC3 (prostate). Between 5,000 and 10,000 cells were seeded in 96 well plates on Day 0 and the compounds were added 1 day later and incubated for 4 days. All compounds were tested in half-log steps at concentrations indicated in Table 1. The activity was assessed by their IC₅₀ and IC₇₀ values.

TABLE 1
Concentration ranges for single agent dose response test

| | | Concentration (ng/mL) | |
|---|---|---|---|
| Cell Line | Drug | Low | High |
| MCF-7 ER-positive breast | 5-FU | 10 | 300,000 |
| cancer | Vinorelbine | 0.003 | 100 |
| | Paclitaxel | 0.003 | 100 |
| | Tamoxifen | 3 | 100,000 |
| | Indibulin | 0.1 | 3,000 |
| A549 NSCLC | Carboplatin | 3 | 100,000 |
| | Etoposide | 3 | 100,000 |
| | Erlotinib | 0.1 | 30,000 |
| | Indibulin | 0.1 | 3,000 |

Growth IC₅₀ and IC₇₀ values (Table 2) were determined using XLfit software package, and used to set the concentration ranges and drug ratios for the synergy detection part of the study.

TABLE 2
Growth IC₅₀ and IC₇₀ values for A549 and MCF-7 cell lines

| | | |
|---|---|---|
| Compound | IC₅₀ (ng/mL) | IC₇₀ (ng/mL) |

Cell Line: A549

| | | |
|---|---|---|
| Carboplatin | 11,470 | 39,200 |
| Etoposide | 52 | 228 |
| Erlotinib | 7860 | >30,000 |
| Gemcitabine | 1.7 | 2.5 |
| Indibulin | 38 | 140 |

Cell Line: MCF7

| Vinorelbine | 0.18 | 0.29 |
|---|---|---|
| Paclitaxel | 0.3 | 1.0 |
| Tamoxifen | 110 | 3540 |
| Indibulin | 110 | 220 |
| Compound | IC₅₀ (μg/mL) | IC₇₀ (μg/mL) |

Cell Line: SKOV3

| | | |
|---|---|---|
| Cisplatin | 2.15 | 5.25 |
| Carboplatin | 27.27 | 69.04 |
| Altretamine | >100 | >100 |
| Gemcitabine | 0.015 | 0.055 |
| Indibulin | 0.061 | 4.66 |

Cell Line: PC3

| | | |
|---|---|---|
| Estramustine | 27.50 | 68.18 |
| Goserelin | >100 | >100 |
| Prednisolone | 185.007 | 287.102 |
| Indibulin | 0.015 | 0.027 |

Chou-Talalay median effect method was used to quantitatively characterize drug-drug interaction [Chou T. C. Theoretical basis, experimental design, and computerized simulation of synergism and antagonism in drug combination studies. Pharmacol Rev. 2006 September; 58(3):621-81]. Test agents were applied to cells at the same time at a constant, equipotent ratio, based on the previously determined respective single agent IC₅₀s (e.g. if IC₅₀(drug A)=0.2 μM and IC₅₀(drug B)=10 μM the ratio is set to 1:50). Mixtures of the drugs at multiples of their single agent IC₅₀'s were prepared, as well as serial dilutions of the mixture. Additionally, single agent dose response was determined for the same concentration ranges as used for combination study.

The coefficient of interaction (CI (IC₅₀)) at IC₅₀ and coefficient of interaction under optimal experimental concentrations (CI (e)) were determined using CalcuSyn software package (Table 3, tests were performed in quadruplicate, mean values reported). Commonly, experimental values of CI<0.8 are considered synergistic, 0.8<CI<1.2 additive.

TABLE 3
Synergy of select chemotherapeutic drugs with indibulin as determined
using Chou-Talalay Coefficient of Interaction (CI) approach
Cell Line: A549

| Carboplatin | | Erlotinib | | Etoposide | | Gemcitabine | |
|---|---|---|---|---|---|---|---|
| CI (IC₅₀) | CI (e) | CI (IC₅₀) | CI (e) | CI (IC₅₀) | CI (e) | CI (IC₅₀) | CI (e) |
| 0.91 | 0.72 | 0.49 | 0.32 | 1.43 | 1.05 | 1.57 | 0.93 |

Cell Line: MCF7

| Vinblastine | | Paclitaxel | | Tamoxifen | | 5-Fluorouracil | | Doxorubicin | |
|---|---|---|---|---|---|---|---|---|---|
| CI (IC₅₀) | CI (e) | CI (IC₅₀) | CI (e) | CI (IC₅₀) | CI (e) | CI (IC₅₀) | CI (e) | CI (IC₅₀) | CI (e) |
| 1.10 | 0.50 | 1.10 | 0.42 | 0.87 | 0.67 | 0.93 | 0.46 | 1.23 | 0.64 |

Cell Line: SKOV3

| Cisplatin | | Carboplatin | | Gemcitabine | |
|---|---|---|---|---|---|
| CI (IC₅₀) | CI (e) | CI (IC₅₀) | CI (e) | CI (IC₅₀) | CI (e) |
| 1.29 | 0.77 | 1.42 | 0.73 | 1.43 | 0.65 |

Cell Line: PC3

| Estramustine | | Prednisolone | |
|---|---|---|---|
| CI (IC₅₀) | CI (e) | CI (IC₅₀) | CI (e) |
| 1.52 | 0.76 | 1.42 | 0.91 |

A549 cells were grown and exposed to indibulin and erlotinib as described above and DNA content measurements with propidium iodide was used to determine the number of viable cells. Concentrations for single agents and combination were as follows (erlotinib:indibulin ratio 208:1):

| | | | | | | | |
|---|---|---|---|---|---|---|---|
| Erlotinib | 988 | 1975 | 3950 | 7900 | 15800 | 31600 | 63200 |
| (ng/mL) | | | | | | | |
| Indibulin | 5 | 9 | 19 | 38 | 76 | 152 | 304 |
| (ng/mL) | | | | | | | |

A549 cells were grown and exposed to indibulin and carboplatin as described above and DNA content measurements with propidium iodide was used to determine the number of viable cells. Concentrations for single agents and combination were as follows (carboplatin:indibulin ratio 303:1):

| | | | | | | | | | | |
|---|---|---|---|---|---|---|---|---|---|---|
| Carboplatin (ng/mL) | 180 | 359 | 719 | 1438 | 2875 | 5750 | 11500 | 23000 | 46000 | 92000 |
| Indibulin (ng/mL) | 0.6 | 1.2 | 2.4 | 4.7 | 9.5 | 19.0 | 38.0 | 75.9 | 151.8 | 303.6 |

MCF-7 cells were grown and exposed to indibulin and 5FU as described above and DNA content measurements with propidium iodide was used to determine the number of viable cells. Concentrations for single agents and combination were as follows (5FU:indibulin ratio 10.9:1):

| | | | | | | | |
|---|---|---|---|---|---|---|---|
| 5-FU (ng/mL) | 15 | 30 | 60 | 120 | 240 | 480 | 960 |
| Indibulin (ng/mL) | 1.4 | 2.8 | 5.5 | 11.0 | 22.0 | 44.0 | 88.1 |

MCF-7 cells were grown and exposed to indibulin and vinorelbine as described above and DNA content measurements with propidium iodide was used to determine the number of viable cells. Concentrations for single agents and combination were as follows (vinorelbine:indibulin ratio 0.016:1):

| | | | | | | | |
|---|---|---|---|---|---|---|---|
| Vinorelbine | 0.023 | 0.045 | 0.090 | 0.180 | 0.360 | 0.720 | 1.440 |
| (ng/mL) | | | | | | | |
| Indibulin | 1.37 | 2.75 | 5.49 | 10.98 | 21.96 | 43.92 | 87.84 |
| (ng/mL) | | | | | | | |

MCF-7 cells were grown and exposed to indibulin and tamoxifen as described above and DNA content measurements with propidium iodide was used to determine the number of viable cells. Concentrations for single agents and combination were as follows (tamoxifen:indibulin ratio 100:1):

| | | | | | | | | | | |
|---|---|---|---|---|---|---|---|---|---|---|
| Tamoxifen (ng/mL) | 17.2 | 34.3 | 68.7 | 137.5 | 275 | 550 | 1100 | 2200 | 4400 | 8800 |
| Indibulin (ng/mL) | 0.17 | 0.34 | 0.69 | 1.38 | 2.75 | 5.50 | 11.00 | 22.00 | 44.00 | 88.00 |

MCF-7 cells were grown and exposed to indibulin and paclitaxel as described above and DNA content measurements with propidium iodide was used to determine the number of viable cells. Concentrations for single agents and combination were as follows (paclitaxel:indibulin ratio 0.27:1):

| | | | | | | |
|---|---|---|---|---|---|---|
| Paclitaxel (ng/mL) | 0.75 | 1.5 | 3 | 6 | 12 | 24 |
| Indibulin (ng/mL) | 2.78 | 5.55 | 11.10 | 22.20 | 44.40 | 88.80 |

Inhibitory Constants (IC) for combination treatment are substantially lower than those for single agents (see Table 4 below).

| Cell Line | Drug | Single Agent (ng/mL) | | | | Combination (ng/mL) | | | |
|---|---|---|---|---|---|---|---|---|---|
| A549 | Indibulin | IC₄₀ | 71 | IC₅₀ | 92 | IC₄₀ | 21 | IC₅₀ | 30 |
| | Erlotinib | IC₄₀ | 20315 | IC₅₀ | 38,525 | | 4424 | | 6200 |
| | Indibulin | IC₇₀ | 95 | IC₅₀ | 54 | IC₇₀ | 27 | IC₅₀ | 11 |
| | Carboplatin | IC₇₀ | 15365 | IC₅₀ | 5,827 | | 8044 | | 3481 |
| | Indibulin | IC₆₀ | 129 | | | IC₆₀ | 68 | | |
| | Etoposide | IC₆₀ | 177 | | | | 94 | | |
| MCF7 | Indibulin | IC₆₀ | 62 | IC₅₀ | 13 | IC₆₀ | 2.8 | IC₅₀ | 5 |
| | 5-FU | IC₆₀ | 84 | IC₅₀ | 92 | | 30 | | 57 |
| | Indibulin | IC₆₀ | 18.1 | IC₅₀ | 13 | IC₆₀ | 6 | IC₅₀ | 8 |
| | Vinorelbine | IC₆₀ | 0.26 | IC₅₀ | 0.19 | | 0.10 | | 0.08 |
| | Indibulin | IC₆₀ | 18.1 | IC₅₀ | 13 | IC₆₀ | 6.4 | IC₅₀ | 4 |
| | Tamoxifen | IC₆₀ | 3166 | IC₅₀ | 1570 | | 641 | | 376 |
| | Indibulin | IC₈₄ | 53 | IC₅₀ | 14 | IC₈₄ | 10 | IC₅₀ | 4 |
| | Paclitaxel | IC₈₄ | 5.3 | IC₅₀ | 1.27 | | 2.7 | | 1.08 |
| | Indibulin | | | IC₅₀ | 13 | | | IC₅₀ | 11 |
| | Doxorubicin | | | IC₅₀ | 7 | | | | 3 |

| | | | | | |
|---|---|---|---|---|---|
| SKOV3 | Indibulin | IC₅₀ | 415 | IC₅₀ | 22 |
| | Cisplatin | IC₅₀ | 773 | | 780 |
| | Indibulin | IC₅₀ | 415 | IC₅₀ | 29 |
| | Carboplatin | IC₅₀ | 12,026 | | 12,881 |
| | Indibulin | IC₅₀ | 415 | IC₅₀ | 47 |
| | Gemcitabine | IC₅₀ | 11 | | 12 |
| PC3 | Indibulin | IC₅₀ | 34 | IC₅₀ | 14 |
| | Estramustine | IC₅₀ | 29,699 | | 26,466 |
| | Indibulin | IC₅₀ | 34 | IC₅₀ | 12 |
| | Prednisolone | IC₅₀ | 137,188 | | 145,659 |

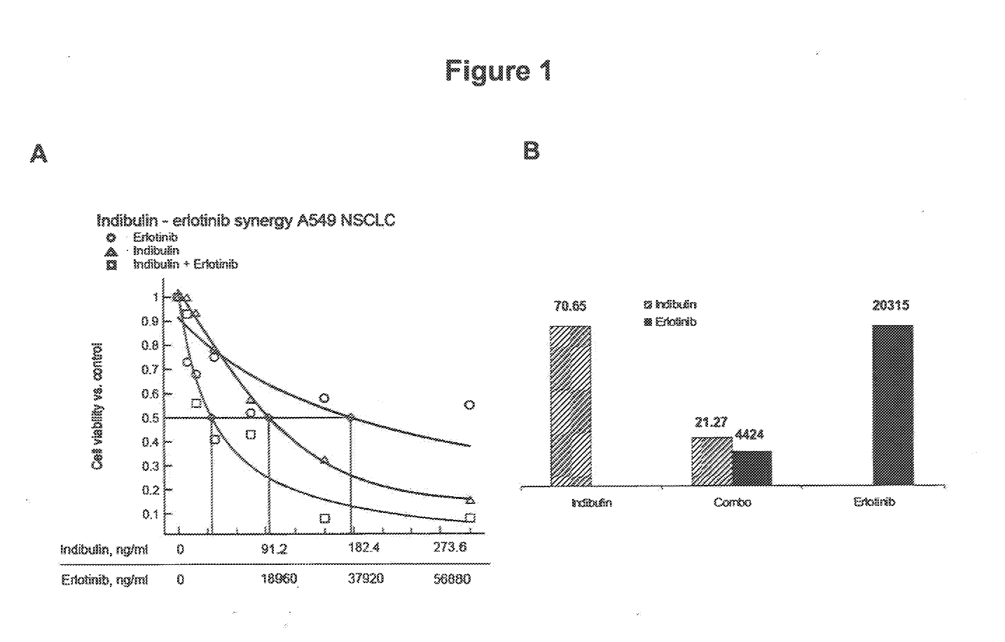

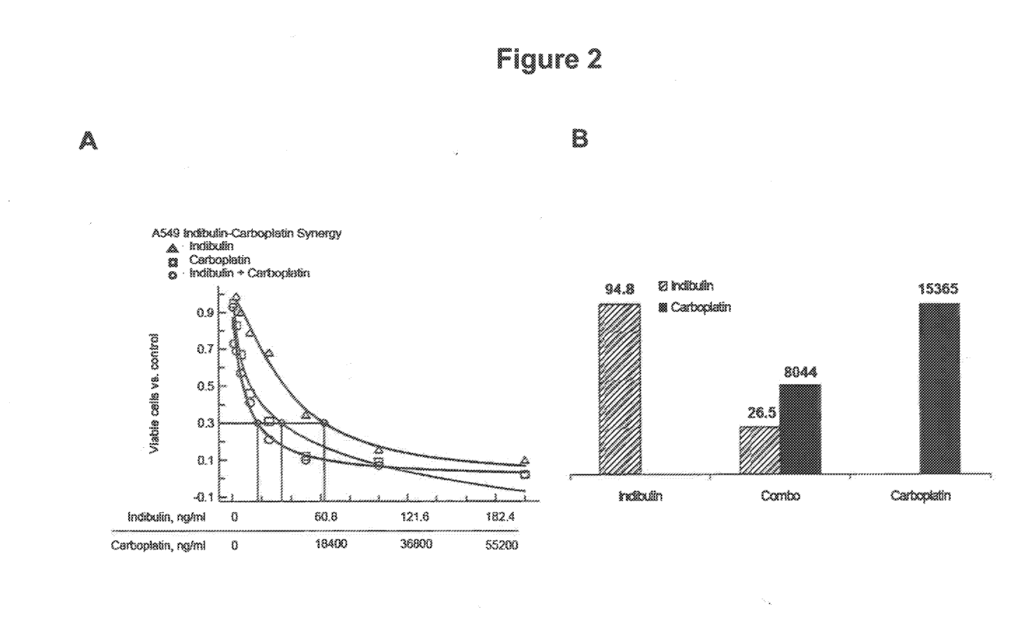

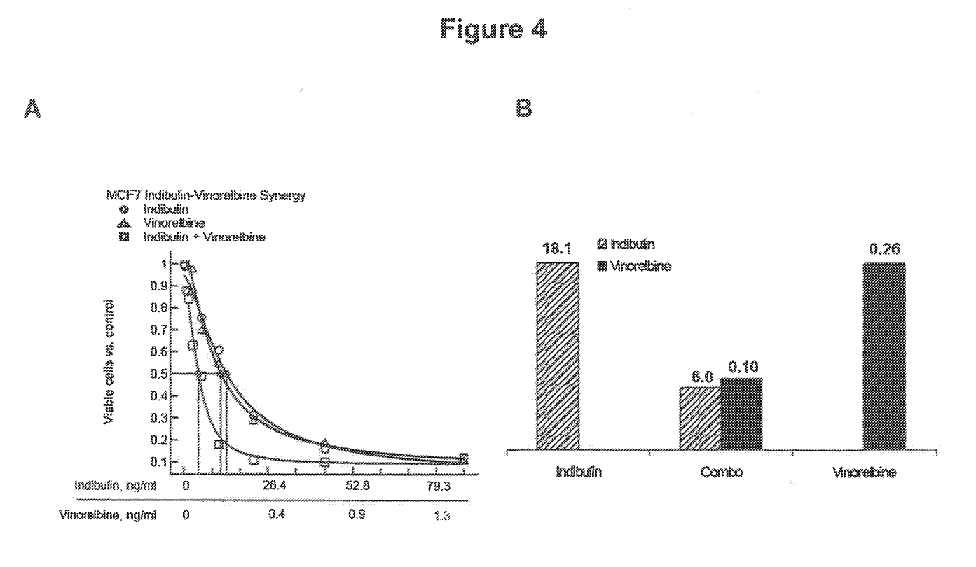

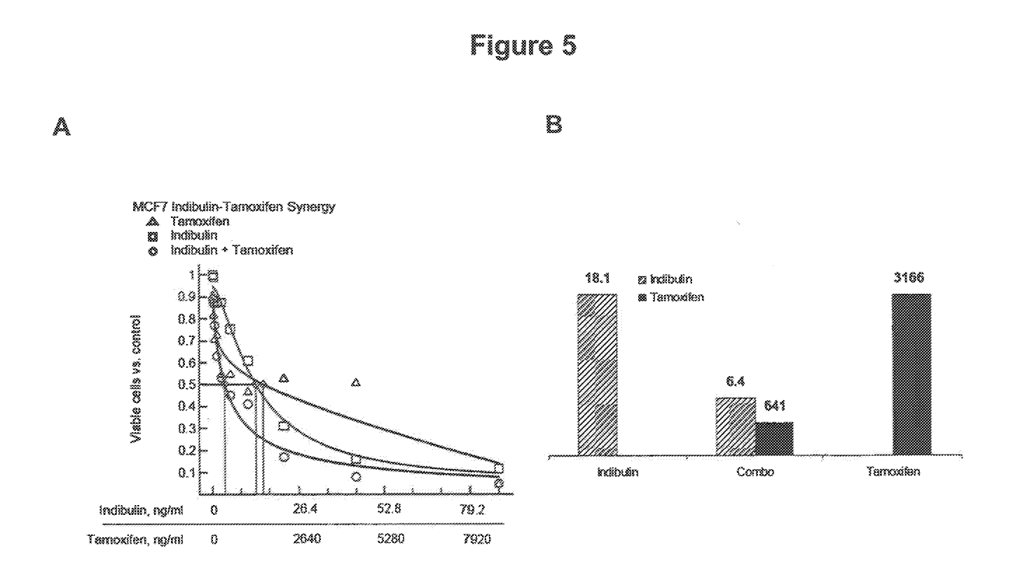

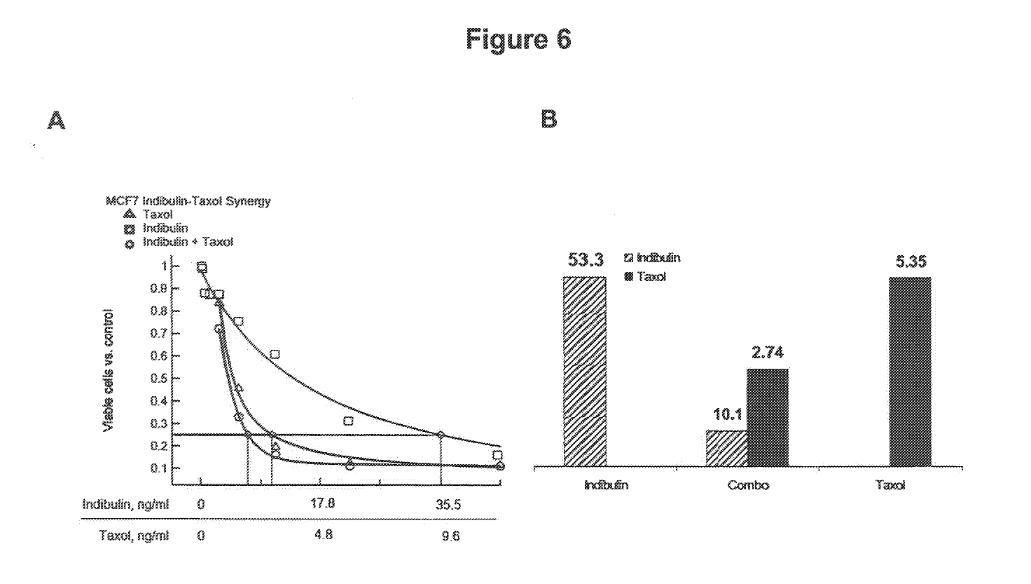

Representative dose response curves for individual drugs and combinations are shown in FIGS. 1-6.

### Tubulin Binding Site

### Example 1

(N-(pyridin-4-yl)-[1-(4-chlorobenzyl)-indol-3-yl]-glyoxylic acid amide; also called indibulin and D-24851) is shown below.

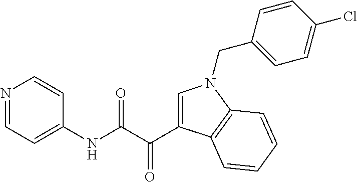 [SMILES: O=C(Nc1ccncc1)C(=O)c1cn(Cc2ccc(Cl)cc2)c2ccccc12]

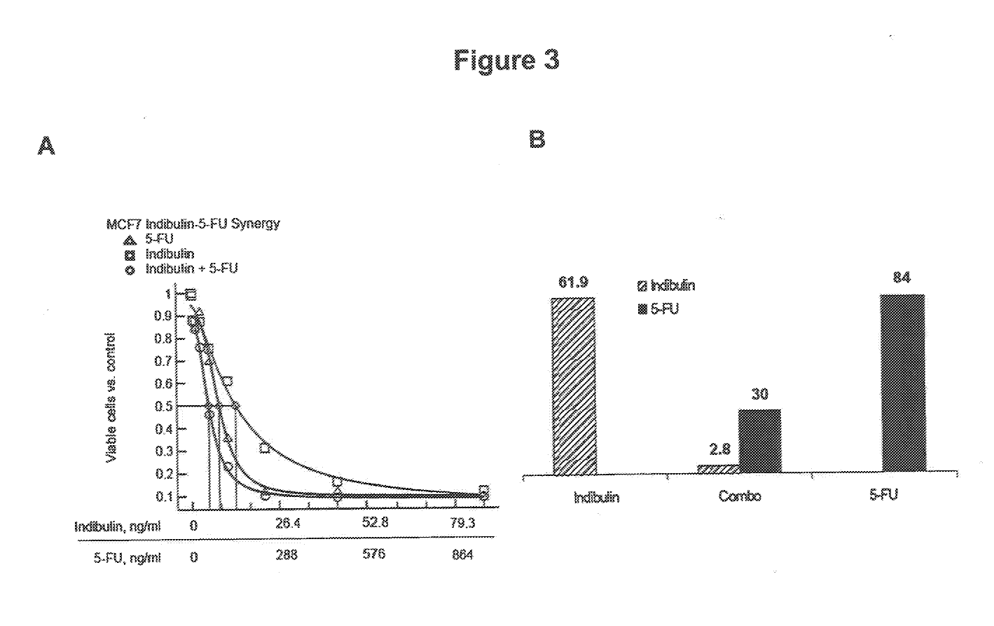

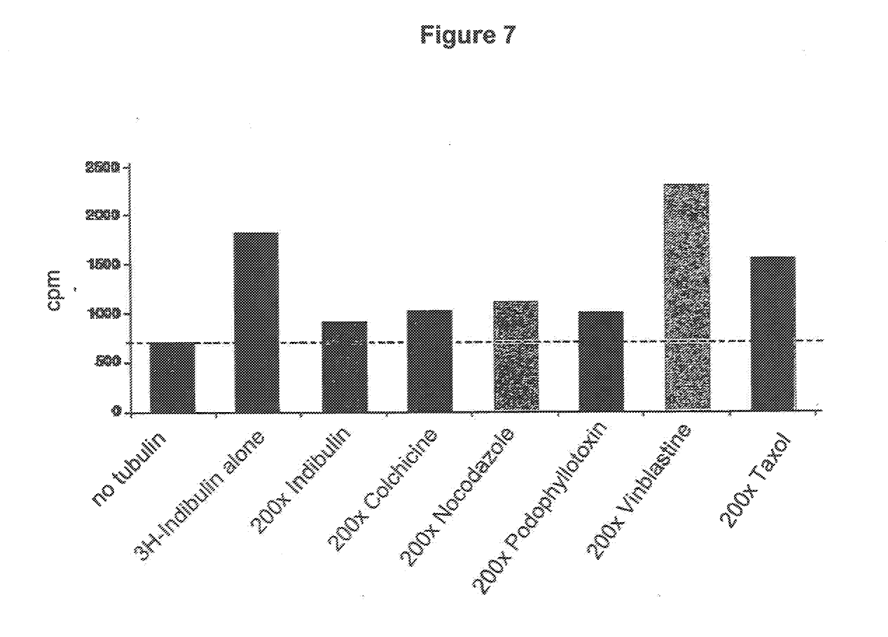

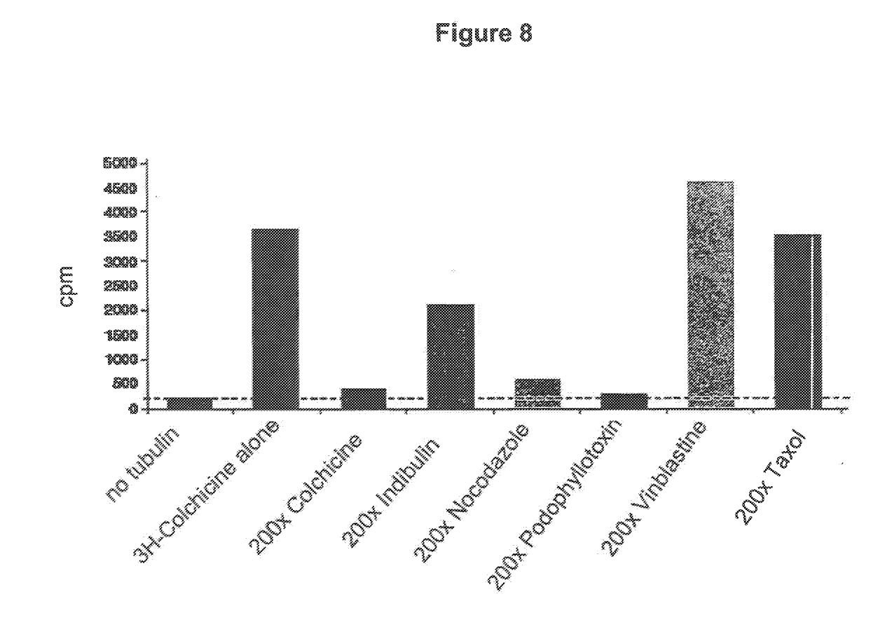

To further define the tubulin binding site of indibulin, tritium labeled indibulin was incubated with purified bovine brain tubulin in the presence or absence of various tubulin binding agents. As shown in FIGS. 7 and 8, 3H-indibulin or 3H-colchicine were incubated with biotin-labeled calf brain tubulin. Where indicated, a 200-fold molar excess of cold competitor was added. Tubulin heterodimers were then precipitated with streptavidin-coated SPA beads and bound radioactivity was determined. “No tubulin” indicates non-specifically bound radioactivity measured in the absence of biotin-labeled tubulin. FIG. 7 shows that colchicine, nocodazole and podophyllotoxin (all bind to the same tubulin binding site) compete with 3H-indibulin for tubulin binding while inblastine and taxol do not compete. FIG. 8 shows that indibulin inhibits about 40% of 3H-colchicine binding. Nocodazole and podophyllotoxin completely inhibit binding while taxol and vinblastine have no effect.

These data and the previously published observation that indibulin does not compete with 3H-colchicine binding in un-biotinylated calf brain tubulin (G. Bacher, Cancer Res. 61, 2001) indicates that the indibulin binding site might be different from but overlapping with colchicine.

### Example 2

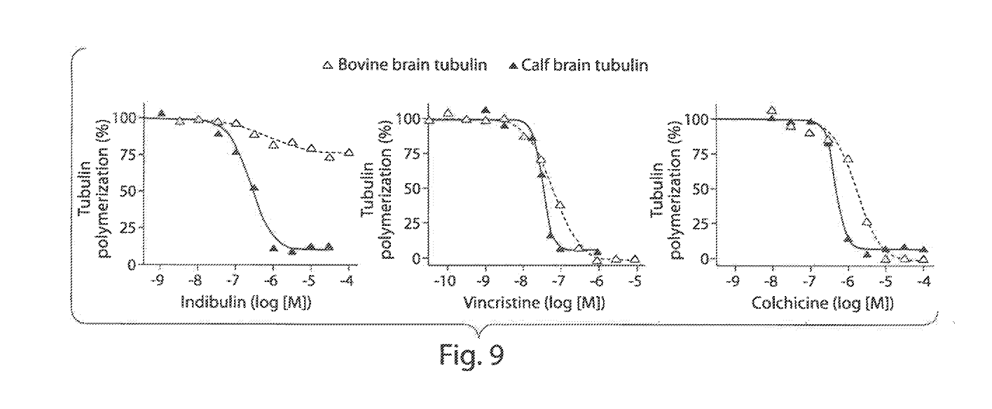

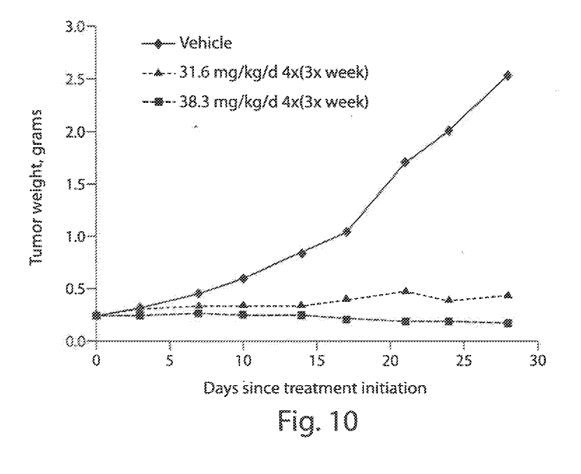

Tubulin from neuronal tissue is post-translationally modified and the degree of modification increases during neuronal development. Calf brain neuronal tubulin and tubulin from other tissues differ in their post-translational modifications from that of adult bovine brain. Polymerization of purified calf brain tubulin was inhibited by increasing concentrations of indibulin, with an IC₅₀ value of about 0.25 μM and a maximum inhibition of 90% as shown in FIG. 9. Tubulin polymerization is given as % over DMSO control. Polymerization of bovine brain tubulin was inhibited by indibulin only by 25% at the highest dose tested. In contrast vincristine or colchicine inhibited both, calf and bovine brain tubulin polymerization to a similar extent. The inability of indibulin to bind to neuronal tubulin supports the observed lack of neurotoxicity with indibulin in preclinical studies as well as in Phase 1 clinical trials.

### Example 3

Indibulin does not disrupt axonal microtubules of rat pheochromocytoma (PC12) cells. The outgrowth of neurites of PC12 was induced by NGF for 5-6 days. Cells were subsequently treated with DMSO, indibulin or colchicine for 24 h (2×IC₅₀ concentration each). Microtubules within the neurites, were visualized by immunostaining with an antibody recognizing acetylated (axonal) tubulin, leaving the cell bodies unstained. DMSO control and indibulin treated cells show identical staining patterns, indicating that indibulin did not affect axonal microtubules. In contrast, treatment with colchicine resulted in a strongly reduced and diffuse staining of microtubules indicating that microtubules were partially disrupted by colchicine.

### Example 4

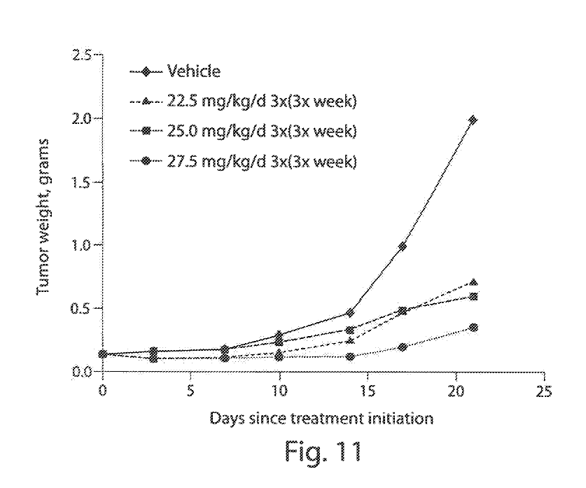

To extend earlier observations of indibulin anti-tumor activity, indibulin was tested in a series of cancer models, including human glioblastoma xenograft (U 87). FIG. 11 shows that orally administered indibulin inhibits growth of U87 glioblastoma xenografts. 5×106 U87 glioblastoma cells were implanted subcutaneously in immunodeficient nu/nu mice. Treatment started when tumor weight was about 0.15 g (Day 0).

### Example 5

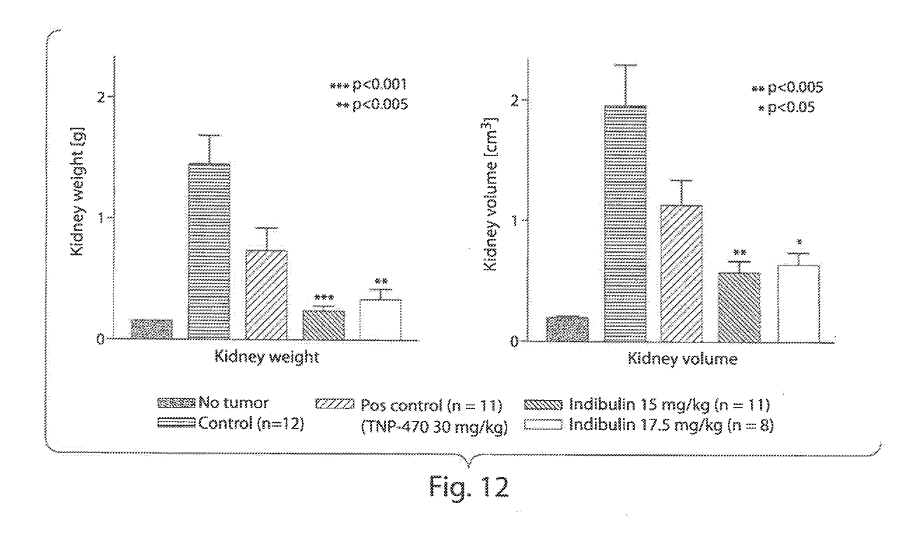

As shown in FIG. 12, orally administered indibulin inhibits growth of murine renal cell carcinoma RENCA. 1.5×106 RENCA cells were injected into the subcapsular space of the kidney through a flank incision (Day 1). Indibulin was orally administered daily at indicated doses from Day 1-20 (5×/week). On Day 21 mice were sacrificed and weight and volume of primary tumors, weight and number of metastases of the lung and metastasis formation in the abdominal lymph nodes determined. Reduction in tumor volume and weight by indibulin (D-24851) was statistically significant. Metastasis data not shown; there was a trend in reduction of number of lung metastases but was not statistically significant. TNP-470 has shown anti-tumor activity in different animal models and is well established for its use in RENCA.

### Example 6

To extend earlier observations of indibulin anti-tumor activity, indibulin was tested in a series of cancer models, including murine renal cell carcinoma (RENCA), DMBA induced mammary carcinoma in rats, human ovarian xenograft (SK-OV-3), human prostate cancer xenograft (PC-3), human vulvar squamous cell carcinoma xenograft (A431), human glioblastoma xenograft (U 87), human mamma carcinoma (MCF-7), and human lung carcinoma (A 549). In all models indibulin demonstrated strong and statistically significant anti-tumor activity. Importantly, at efficacious doses animals showed no signs of neurotoxicity or loss of body weight associated with treatment with taxanes or vinca alkaloids. The significant differences in pharmacodynamics and safety profile of indibulin make it a strong candidate for development as an anti-cancer drug.

| | Organ or | Species of | Dosing | Growth |
|---|---|---|---|---|
| Tumor | Origin | Origin | Schedule | Inhibition(%)* |
| MCF7 | Breast | Human | 38.3 mg/kg/d, | >100 |
| | | | 4x(3x week) | |
| DMBA | Breast | Rat | 46.4 mg/kg/d, | 99.5 |
| | | | 2x(3x week) | |
| U87 | Glial | Human | 27.5 mg/kg/d, | 88.8 |
| | | | 3x(3x week) | |
| RENCA | Renal | Mouse | 17.5 mg/kg/d, | 90 |
| | | | d. 1-20(5x | |
| | | | week) | |
| PC-3 | Prostate | Human | 180 mg/kg/d, d. | 63 |
| | | | 0, 7, 14 | |
| A431 | Vulva | Human | 27.5 mg/kg/d, | 63 |
| | | | 3x(3x week) | |
| SKOV-3 | Ovary | Human | 100 mg/kg/d, d. | 53 |
| | | | 0, 7, 14 | |

*At sacrifice of control group animals

### Example 7

Indibulin concentrations that exhibit antiangiogenic activity in preclinical models were well within plasma concentrations observed in ongoing Phase I studies. In a recently initiated study in the US, three patients were treated with 400 mg BID of indibulin on a continuous treatment schedule. One patient, a 76-year-old man diagnosed with papillary thyroid cancer showed stable disease after treatment; however, tumor measurements decreased 11% at first assessment and thyroglobulin level decreased 34% from baseline and after 15th day of dosing. Treatment is ongoing. Additionally, a 58-year-old woman diagnosed with ovarian cancer with brain metastases had a CA125 level reduced by 11% from baseline after 22 days of treatment with indibulin. Treatment is ongoing.

### EQUIVALENTS

Those skilled in the art will recognize, or be able to ascertain using no more than routine experimentation, numerous equivalents to the compounds and methods of use thereof described herein. Such equivalents are considered to be within the scope of this invention and are covered by the following claims.

All of the above-cited references and publications are hereby incorporated by reference. References incorporated herein by reference in their entirety include, but are not limited to, WO 2006/133835, WO 2006/052712, US 2004/0171668, and 2003/0195360.

We claim:

1. A method for treating cancer, comprising administering indibulin or a pharmaceutically acceptable salt thereof; and one or more other therapeutic agents, wherein the combination shows efficacy that is greater than the efficacy of either agent being administered alone.

2. The method of claim 1, wherein the indibulin or a pharmaceutically acceptable salt thereof is administered orally or intravenously.

3. (canceled)

4. The method of claim 1, wherein the efficacy of indibulin and the one or more other therapeutic agents are synergistic or additive.

5. (canceled)

6. The method of claim 1, wherein the other therapeutic agent is selected from erlotinib, carboplatin, 5-fluorouracil, capecitabine, paclitaxel, tamoxifen, vinorelbine, cisplatin, gemcitabine, estramustine, doxorubicin, vinblastine, etoposide, and prednisolone.

7. The method of claim 1, wherein the cancer is selected from lung, breast, ovarian, and prostate cancer.

8. The method of claim 1, wherein the compound and the one or more other therapeutic agents are administered simultaneously.

9. (canceled)

10. (canceled)

11. A kit comprising indibulin and another therapeutic agent selected from erlotinib, carboplatin, 5-fluorouracil, capecitabine, paclitaxel, tamoxifen, vinorelbine, cisplatin, gemcitabine, estramustine, doxorubicin, vinblastine, etoposide, and prednisolone.

12. A method for treating cancer, comprising administering an indolyl-3-glyoxylic acid derivative of Formula (I) or a pharmaceutically acceptable salt thereof and one or more other therapeutic agents, wherein the combination shows efficacy that is greater than the efficacy of either agent being administered alone 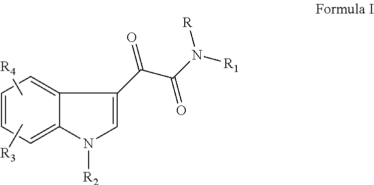
wherein
R is selected from hydrogen; (C₁-C₆)-alkyl, where the alkyl group is optionally mono- or polysubstituted with a phenyl ring which is optionally mono- or polysubstituted with halogen, (C₁-C₆)-alkyl, (C₃-C₇)-cycloalkyl, carboxyl, carboxyl esterified with (C₁-C₆)-alkanol, trifluoromethyl, hydroxyl, methoxy, ethoxy, benzyloxy or a benzyl group which is mono- or polysubstituted on the phenyl moiety with (C₁-C₆)-alkyl groups, halogen or trifluoromethyl; benzyloxycarbonyl; tertiary-butoxycarbonyl; and acetyl;
R₁ is selected from a phenyl ring, which is optionally mono- or polysubstituted with (C₁-C₆)-alkyl, (C₁-C₆)-alkoxy, cyano, halogen, trifluoromethyl, hydroxyl, benzyloxy, nitro, amino, (C₁-C₆)-alkylamino, (C₁-C₆)-alkoxycarbonylamino, carboxyl, or by carboxyl esterified with C₁-C₆-alkanol; a pyridine structure of the Formula (II) 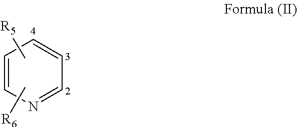
or an N-oxide thereof, where the pyridine structure is alternatively bonded to the ring carbon atoms 2, 3 or 4 and is optionally substituted with the substituents R₅ and R₆, wherein R₅ and R₆ are identical or different and are selected from (C₁-C₆)-alkyl, (C3-C7)-cycloalkyl, (C₁-C₆)—alkoxy, nitro, amino, hydroxyl, halogen, trifluoromethyl, ethoxycarbonylamino, and carboxyalkyloxy in which the alkyl group comprises 1-4 C atoms; 2- or 4-pyrimidinyl, wherein the 2-pyrimidinyl ring is optionally mono- or polysubstituted with a methyl group; 2-, 3-, 4- or 8-quinolyl structure which is optionally substituted with (C₁-C₆)-alkyl, halogen, nitro, amino or (C₁-C₆)-alkylamino; 2-, 3-, or 4-quinolylmethyl group, where the ring carbons of the pyridylmethyl, the quinolyl, and the quinolylmethyl are optionally substituted with (C₁-C₆)-alkyl, (C₁-C₆)-alkoxy, nitro, amino or (C₁-C₆)-alkoxycarbonylamino; and allylaminocarbonyl-2-methylprop-1-yl;
R₁ in the case in which R is hydrogen, methyl, benzyl, benzyloxycarbonyl, tert-butoxycarbonyl, or acetyl, is further selected from —CH₂COOH; —CH(CH₃)—COOH; —(CH₃)₂—CH—(CH₂)₂—CH—COO—; H₃C—H₂C—CH(CH₃)—CH(COOH)—; HO—H₂C—CH(COOH)—; phenyl-CH₂—CH(COOH)—; (4-imidazolyl)-CH₂—CH—(COOH)—; HN═(NH₂)—NH—(CH₂)₃—CH(COOH)—; H₂N—(CH₂)₄—CH(COOH)—; H₂N—CO—CH₂—CH—(COOH)—; and HOOC—(CH₂)₂—CH(COOH)—;
R₁, in the case in which R is hydrogen, benzyloxycarbonyl, tert-butoxycarbonyl, acetyl or benzyl, may be the acid radical of a natural or unnatural amino acid (e.g. α-glycyl, α-sarcosyl, α-seryl, α-phenylalanyl, α-histidyl, α-prolyl, α-arginyl, α-lysyl, α-asparagyl or α-glutamyl), where the amino groups of the respective amino acids may be protected or unprotected, wherein suitable protecting groups include, but are not limited to, benzyloxycarbonyl, tert-butoxycarbonyl, or acetyl, and in the case where R₁ is asparagyl or glutamyl, the second, unbonded carboxyl group is present as a free carboxyl group or in the form of an ester of a C₁-C₆-alkanol (e.g. as a methyl, ethyl or as a tert-butyl ester);
R and R₁ can further form, together with the nitrogen atom to which they are bonded, a piperazine ring of the Formula (III) or a homopiperazine ring, provided R₁ is an aminoalkylene group, in which 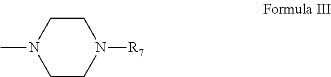
R₇ is selected from alkyl; phenyl which is optionally mono- or polysubstituted with (C₁-C₆)-alkyl, (C₁-C₆)-alkoxy, halogen, nitro, amino or by (C₁-C₆)-alkylamino; benzhydryl and bis-p-fluorobenzhydryl;
R₂ is selected from hydrogen; (C₁-C₆)-alkyl, wherein the alkyl group is optionally mono- or polysubstituted with halogen, phenyl (wherein the phenyl is optionally mono- or polysubstituted with halogen, (C₁-C₆)-alkyl, (C3-C7)-cycloalkyl, carboxyl, carboxyl esterified with C₁-C₆-alkanol, trifluoromethyl, hydroxyl, methoxy, ethoxy or benzyloxy), 2-quinolyl (optionally mono- or polysubstituted with halogen, (C₁-C₄-alkyl or (C₁-C₄)-alkoxy), or 2-, 3- or 4-pyridyl (optionally mono- or polysubstituted with halogen, (C₁-C₄)-alkyl or (C₁-C₄)-alkoxy); aroyl (where the aryl moiety is optionally mono- or polysubstituted with halogen, (C₁-C₆)-alkyl, (C₃-C₇)-cycloalkyl, carboxyl, carboxyl esterified with C₁-C₆-alkanol, trifluoromethyl, hydroxyl, methoxy, ethoxy or benzyloxy);
R₃ and R₄ are identical or different and are selected from hydrogen, (C₁-C₆)-alkyl, (C₃-C₇)-cycloalkyl, (C₁-C₆)-alkanoyl, (C₁-C₆)-alkoxy, halogen, benzyloxy, nitro, amino, (C₁-C₄)-mono or dialkyl-substituted amino, (C₁-C₆)-alkoxycarbonylamino and (C₁-C₆)-alkoxycarbonylamino-(C₁-C₆)-alkyl;
Z is O or S.

13. The method of claim 12, wherein R₂ is selected from (C₁-C₆)-alkyl, wherein the alkyl group is optionally mono- or polysubstituted with halogen, phenyl (wherein the phenyl is optionally mono- or polysubstituted with halogen, (C₁-C₆)-alkyl, (C₃-C₇)-cycloalkyl, carboxyl, carboxyl esterified with C₁-C₆-alkanol, trifluoromethyl, hydroxyl, methoxy, ethoxy or benzyloxy), 2-quinolyl (optionally mono- or polysubstituted with halogen, (C₁-C₄)-alkyl or (C₁-C₄)-alkoxy), or 2-, 3- or 4-pyridyl (optionally mono- or polysubstituted with halogen, (C₁-C₄)-alkyl or (C₁-C₄)-alkoxy); aroyl (where the aryl moiety is optionally mono- or polysubstituted with halogen, (C₁-C₆)-alkyl, (C₃-C₇)-cycloalkyl, carboxyl, carboxyl esterified with C₁-C₆-alkanol, trifluoromethyl, hydroxyl, methoxy, ethoxy or benzyloxy).

14. The method of claim 12, wherein the other therapeutic agent is selected from erlotinib, carboplatin, 5-fluorouracil, capecitabine, paclitaxel, tamoxifen, vinorelbine, cisplatin, gemcitabine, estramustine, doxorubicin, vinblastine, etoposide, and prednisolone.

15. (canceled)

16. The method of claim 12, wherein the indolyl-3-glyoxylic acid derivative is indibulin.

17. The method of claim 24, further comprising administering the indolyl-3-glyoxylic acid derivative in combination with another agent or therapy method.

18. The method of claim 17, wherein other agent or therapy is selected from a chemotherapeutic, radiotherapy, hormonal therapeutic agents, targeted therapy, immunotherapy, gene therapy, or surgery.

19. The method of claim 18, wherein the other agent is a chemotherapeutic.

20. The method of claim 17, wherein the individual components of the combination are administered simultaneously, sequentially, or separately.

21. The method of claim 17, wherein the indolyl-3-glyoxylic acid derivative is indibulin.

22. method of claim 17, wherein the cancer is selected from adenoid cystic carcinoma, renal cell carcinoma, breast cancer, ovarian cancer, prostate cancer, vulvar cancer, glioblastoma, and lung cancer.

23. The method of claim 22, wherein the cancer is selected from renal cell carcinoma, breast cancer, vulvar cancer, glioblastoma, and lung cancer.

24. A method for the treatment of cancer, comprising administering an indolyl-3-glyoxylic acid derivative with a daily dose of 100 to 2000 mg.

25. (canceled)

26. The method of claim 24, wherein the indolyl-3-glyoxylic acid derivative is administered once daily, twice daily, or as a continuous treatment.

27. (canceled)

28. (canceled)
